(19) United States
(12) Patent Application Publication
(10) Pub. No.: US 2010/0247538 A1
(43) Pub. Date: Sep. 30, 2010

(54) IL-18 and Protein Kinase R Inhibition for the Treatment of COPD

(75) Inventors: Jack A. Elias, New Haven, CT (US); Chun Geun Lee, Orange, CT (US); Min-Jong Kang, Woodbridge, CT (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Yale University

(21) Appl. No.: 12/601,778

(22) PCT Filed: May 29, 2008
(86) PCT No.: PCT/US08/06821

§ 371 Date: Jun. 2, 2010

Related U.S. Application Data
(60) Provisional application No. 60/932,158, filed on May 29, 2007.

(51) Int. Cl. A61K 39/395; A61K 31/713; A61K 31/7088; A61K 31/7105; A61K 38/02; A61P 11/00

(52) U.S. Cl. 424/135.1; 424/130.1; 424/141.1; 424/133.1; 514/44 A; 514/44 R; 514/18.9; 514/1.1

(57) ABSTRACT

The present invention encompasses compositions and methods directed to preventing, inhibiting, or treating inflammation, alveolar remodeling, or cell death in lung which results from elevation of IL-18, IFN-γ, or PKR where the methods of the present invention comprise administering an IL-18 inhibitor, an IL-18Rα inhibitor, and IFNγ inhibitor, a PKR inhibitor, and any combination thereof to an individual experiencing inflammation, alveolar remodeling, or cell death in lung. The present invention further encompasses a method of alleviating exacerbations of COPD frequently caused by viral infection by administering inflammation, alveolar remodeling, or cell death in lung to a patient with COPD at risk of developing a viral infection or who has acquired a viral infection.

### BACKGROUND OF THE INVENTION

Chronic obstructive pulmonary disease (COPD) is the fourth leading cause of death in the United States, claiming the lives of 122,283 Americans in 2003 alone. In 2004, 11.4 million adults over the age of 18 were estimated to have COPD. However, over 24 million U.S. adults have evidence of impaired lung function, indicating an under-diagnosis of COPD (American Lung Association, 2006, Trends in Chronic Bronchitis and Emphysema Morbidity and Mortality). The cost to the nation for COPD in 2004 was approximately $37.2 billion dollars, including healthcare expenditures of $20.9 billion in direct health care expenditures, $7.4 billion in indirect morbidity costs, and $8.9 billion in indirect mortality costs (American Lung Association, 2006, Trends in Chronic Bronchitis and Emphysema Morbidity and Mortality).

Smoking is responsible for 90% of COPD in the United States. Although not all cigarette smokers will develop COPD, it is estimated that 15% will. Smokers with COPD have higher death rates than nonsmokers with COPD. They also have more frequent respiratory symptoms (coughing, shortness of breath, etc.) and more deterioration in lung function than non-smokers.

COPD is comprised primarily of two related diseases: chronic bronchitis and emphysema. Chronic bronchitis is the inflammation and eventual scarring of the lining of the bronchial tubes. When the bronchi are inflamed and/or infected, less air is able to flow to and from the lungs and a heavy mucus or phlegm is coughed up. The condition is defined by the presence of a mucus-producing cough most days of the month, three months of a year for two successive years without other underlying disease to explain the cough. Emphysema begins with the destruction of air sacs (alveoli) in the lungs where oxygen from the air is exchanged for carbon dioxide in the blood, due in part, by an abnormal inflammatory response of the lung to noxious particles or gases, chiefly cigarette smoke. The walls of the air sacs are thin and fragile. Damage to the air sacs is irreversible and results in permanent “holes” in the tissues of the lower lungs. As air sacs are destroyed, the lungs are able to transfer less and less oxygen to the bloodstream, causing shortness of breath. The lungs also lose their elasticity, which is important to keep airways open. As a result, the patient experiences great difficulty exhaling. In both chronic bronchitis and emphysema the obstruction is generally permanent and progressive.

COPD patients often experience exacerbations. The term “exacerbation” refers to the aggravation of the symptoms or an increase in the severity of the disease. The duration of an exacerbation can vary greatly—from hours to several days. Exacerbations often require a call or visit to the clinician, an emergency room visit, and a possible change in medication. An exacerbation is defined as a complex of respiratory events reported as adverse events, with a duration of 3 or more days.

Exacerbations may cause symptoms specific to the respiratory process to increase. The patient may experience increased dyspnea, a productive cough with an altered sputum, and fever. While a patient's dyspnea may vary day to day when the COPD is stable, the dyspnea present in an exacerbation exceeds the worst of those variations. The sputum may increase or be more purulent and change color. The patient may also experience nonspecific symptoms such as malaise, fatigue, insomnia, sleepiness, or depression. Exacerbations of COPD are usually caused by an infection of the lower respiratory tract. The most common causes of infection are: aerobic Gram-positive and Gram-negative bacteria, atypical bacteria, respiratory virus, rhinovirus, influenza virus, RSV, or a combination of pathogens. Viral exacerbations are more severe, last longer, and are assocated with greater levels of inflammation and loss of lung function than exacerbations due to other causes (Wedzicha, 2004, PATS; Seemungel et al., 2001, AM. J. RCCM; Tan et al., 2003, Am. J. Med. Donaldson et al., 2000, Thorax).

Each COPD patient is likely to experience 1 to 4 exacerbations a year, or a total of 15 to 16 million episodes in the US alone. While many patients experience these exacerbations, it is estimated that they only report about 50% of all episodes to physicians. Frequent exacerbations have been associated with a poor quality of life and a high economic burden.

In addition, influenza infections are more severe in cigarette smokers (Arcavi and Benowitz, 2004, Arch Int. Med.; Kark et al., 1982, New Eng,. J. Med.)

The complex etiology and mechanisms of disease progression in COPD patients is poorly understood as are the mechanisms that underlie these virus-induced responses. There is urgent need for new models and the identification of new therapeutic modalities. The present invention fulfills this unmet need.

### SUMMARY OF THE INVENTION

In one embodiment, the present invention encompasses a method of treating a disease associated with dysregulation of IL-18 expression in lung, the method comprising administering a therapeutically effective amount of at least one IL-18 inhibitor to a mammal having the disease wherein the IL-18 inhibitor attenuates, prevents, or halts the dysregulation of IL-18 expression, thereby reducing IL-18 expression in the lungs of the mammal. In one aspect, said IL-18 inhibitor comprises an inhibitor selected from the group consisting of an antibody, siRNA, a ribozyme, an antisense, an aptamer, a peptidomimetic, a small molecule, and any combination thereof. In another aspect, the antibody comprises an antibody selected from the group consisting of a polyclonal antibody, a monoclonal antibody, a humanized antibody, a synthetic antibody, a heavy chain antibody, a human antibody, and a biologically active fragment of an antibody. In another aspect, the mammal is a human.

In another embodiment, the present invention comprises a method of treating a disease associated with dysregulation of IFNγ expression in lung, the method comprising administering a therapeutically effective amount of at least one IFNγ inhibitor to a mammal having the disease wherein the IFNγ inhibitor attenuates, prevents, or halts the dysregulation of IFNγ expression, thereby reducing IFNγ expression in the lungs of said mammal. In one aspect, the IFNγ inhibitor comprises an inhibitor selected from the group consisting of an antibody, siRNA, a ribozyme, an antisense, an aptamer, a peptidomimetic, a small molecule, and any combination thereof. In another aspect, the antibody comprises an antibody selected from the group consisting of a polyclonal antibody, a monoclonal antibody, a humanized antibody, a synthetic antibody, a heavy chain antibody, a human antibody, and a biologically active fragment of an antibody. In another aspect, the mammal is a human.

In still another embodiment, the present invention comprises a method of treating a disease associated with dysregulation of double stranded RNA-dependent protein kinase (PKR) expression in lung, the method comprising administering a therapeutically effective amount of at least one PKR inhibitor to a mammal having the disease wherein said PKR inhibitor attenuates, prevents, or halts the dysregulation of PKR expression, thereby reducing PKR expression in the lungs of the mammal. In one aspect, the PKR inhibitor comprises an inhibitor selected from the group consisting of an antibody, siRNA, a ribozyme, an antisense, an aptamer, a peptidomimetic, a small molecule, and any combination thereof. In another aspect, the antibody comprises an antibody selected from the group consisting of a polyclonal antibody, a monoclonal antibody, a humanized antibody, a synthetic antibody, a heavy chain antibody, a human antibody, and a biologically active fragment of an antibody. In still another aspect, the mammal is a human.

In yet another embodiment, the present invention comprises a method of inhibiting inflammation in the lung of a mammal at risk of developing inflammation, wherein the inflammation is the result of exposure to viral infection and cigarette smoke, the method comprising administering a therapeutically effective amount of an inhibitor to the mammal having the inflammation, wherein the inhibitor prevents the inflammation, and further wherein said inhibitor is selected from the group consisting of an IL-18 inhibitor, an IL-18Rα inhibitor, and IFNγ inhibitor, a PKR inhibitor, and any combination thereof. In one aspect, the inhibitor comprises an inhibitor selected from the group consisting of an antibody, siRNA, a ribozyme, an antisense, an aptamer, a peptidomimetic, a small molecule, and any combination thereof. In another aspect, the antibody comprises an antibody selected from the group consisting of a polyclonal antibody, a monoclonal antibody, a humanized antibody, a synthetic antibody, a heavy chain antibody, a human antibody, and a biologically active fragment of an antibody. In still another aspect, the mammal is a human.

In still another embodiment, the present invention comprises a method of inhibiting alveolar remodeling in the lung of a mammal at risk of developing alveolar remodeling wherein the alveolar remodeling is the result of exposure to viral infection and cigarette smoke, said method comprising administering a therapeutically effective amount of an inhibitor to the mammal having the alveolar remodeling, wherein the inhibitor prevents the alveolar remodeling, and further wherein the inhibitor is selected from the group consisting of an IL-18 inhibitor, an IL-18Rα inhibitor, and IFNγ inhibitor, a PKR inhibitor, and any combination thereof. In one aspect, the inhibitor comprises an inhibitor selected from the group consisting of antibody, siRNA, a ribozyme, an antisense, an aptamer, a peptidomimetic, a small molecule, and any combination thereof. In another aspect, the antibody comprises an antibody selected from the group consisting of a polyclonal antibody, a monoclonal antibody, a humanized antibody, a synthetic antibody, a heavy chain antibody, a human antibody, and a biologically active fragment of an antibody. In still another aspect, the mammal is a human.

In another embodiment, the present invention comprises a method of inhibiting cellular apoptosis in the lung of a mammal at risk of developing cellular apoptosis, wherein the cellular apoptosis is the result of exposure to viral infection and cigarette smoke, the method comprising administering a therapeutically effective amount of an inhibitor to the mammal having cellular apoptosis, wherein the inhibitor prevents the cellular apoptosis, and further wherein the inhibitor is selected from the group consisting of an IL-18 inhibitor, an IL-18Rα inhibitor, and IFNγ inhibitor, a PKR inhibitor, and any combination thereof. In one aspect, the inhibitor comprises an inhibitor selected from the group consisting of an antibody, siRNA, a ribozyme, an antisense, an aptamer, a peptidomimetic, a small molecule, and any combination thereof. In another aspect, the antibody comprises an antibody selected from the group consisting of a polyclonal antibody, a monoclonal antibody, a humanized antibody, a synthetic antibody, a heavy chain antibody, a human antibody, and a biologically active fragment of an antibody. In still another aspect, the mammal is a human.

In yet another embodiment, the present invention comprises a method of treating COPD in a mammal diagnosed with COPD, wherein the COPD comprises inflammation, alveolar remodeling, or cellular apoptosis, the method comprising administering a therapeutically effective amount of an inhibitor to the mammal having COPD, wherein the inhibitor prevents the inflammation, alveolar remodeling, or cellular apoptosis, and further wherein the inhibitor is selected from the group consisting of an IL-18 inhibitor, an IL-18Rα inhibitor, and IFNγ inhibitor, a PKR inhibitor, and any combination thereof. In one aspect, the inhibitor comprises an inhibitor selected from the group consisting of an antibody, siRNA, a ribozyme, an antisense, an aptamer, a peptidomimetic, a small molecule, and any combination thereof. In another aspect, the antibody comprises an antibody selected from the group consisting of a polyclonal antibody, a monoclonal antibody, a humanized antibody, a synthetic antibody, a heavy chain antibody, a human antibody, and a biologically active fragment of an antibody. In still another aspect, the mammal is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1., comprising FIGS. 1A through 1F, is a series of images depicting the inflammatory effects of Poly(I:C) in mice exposed to RA or CS. C57BL/6J mice were exposed to CS or RA (non-smoking, NS) for 2 weeks and then randomized to receive 4 doses of Poly(I:C) (PolyI:C+) or vehicle control (PolyI:C−). The effects of 4 doses of varying concentrations of Poly(I:C) (pIC) on BAL total cell recovery (FIG. 1A), 4 doses of Poly(I:C) (50 μg) on parenchymal (FIG. 1B, magnification ×20) and airway (FIG. 1C, magnification ×20) inflammation and BAL differential cell recovery (FIG. 1D) are illustrated. FIG. 1E depicts the effects of 4 doses of Poly(I:C) on Balb/c mice breathing RA (CS−) and mice exposed to CS (CS+). FIG. 1F illustrates the dose-dependence of these inflammatory events in C57BL/6J mice. The values in FIGS. 1A, 1D, 1E and 1F represent the mean+SEM of evaluations in a minimum of 5 mice. FIGS. 1B and 1C are representative of a minimum of 4 similar experiments (*p<0.05, **p<0.01, ***p<0.001).

FIG. 2, comprising FIGS. 2A through 2G, is a series of images demonstrating the effects of Poly(I:C) and other innate immunity agonists in mice exposed to RA or CS. Mice were exposed to CS or RA (non-smoking, NS) for 2 weeks and then randomized to receive 4 doses of Poly(I:C) (50 μg) (PolyI:C+), LPS (1-10 μg), GDQ (5-50 μg) or vehicle controls. The alterations in alveolar structure, alveolar chord length and lung volume caused by Poly(I:C) are shown in FIGS. 2A (magnification ×4), 2B and 2C. The effects of these interventions on matrix accumulation were assessed with trichrome evaluations (FIG. 2D, magnification ×20). The effects of LPS and GDQ on BAL inflammation are shown in FIGS. 2E and 2F. The effects of LPS and GDQ on alveolar remodeling are illustrated in FIGS. 2G and 2H respectively. The values in FIGS. 2B, 2C and 2E-2H represent the mean+SEM of evaluations in a minimum of 5 mice. FIGS. 2A and 2C are representative of a minimum of 4 similar experiments (*p<0.05, **p<0.01).

FIG. 3, comprising FIGS. 3A through 3E, is a series of images demonstrating the effects of Poly(I:C) on cytokines and IFNs. Mice were exposed to CS or RA for 2 weeks and then randomized to receive Poly(I:C) (50 μg) or vehicle control. The levels of IL-18, IFN-α/β, IL-12/23 p40 and IFN-γ in vehicle-treated mice breathing RA or CS were near or below the limits of detections of these assays. The effects of Poly(I:C) in mice exposed to CS (solid circle) or RA (open circle) are illustrated (FIGS. 3A-3D). The localization of IL-18 was accomplished using IHC (FIG. 3E, magnification ×20). In the lungs from mice exposed to CS plus Poly (I:C), selected IL-18 containing macrophages are highlighted with arrows and a high power image can be seen in the insert (magnification ×100). The values in FIGS. 3A-3D represent the mean+SEM of evaluations in a minimum of 5 mice. FIG. 3E is representative of 3 similar experiments. (*p<0.05, ***p<0.001).

FIG. 4, comprising FIGS. 4A through 4C, is a series of images demonstrating the roles of IL-18Rα and IFN-γ in the interaction of CS and Poly(I:C). WT (+/+) mice and mice with null mutations (−/−) of IL-18Rα or IFN-γ were exposed to CS or RA (CS−) for 2 weeks and then given 4 doses of Poly(I:C) (50 μg) or vehicle control. BAL total cell recovery (FIG. 4A), BAL differential cell recovery (FIG. 4B) and emphysema (FIG. 4C) were evaluated. The noted values represent the mean+SEM of evaluations in a minimum of 5 mice (*p<0.05, **p<0.01).

FIG. 5, comprising FIGS. 5A through 5C, is a series of images demonstrating the roles of IL-18Rα and IFN-γ in CS plus Poly(I:C)-induced cytokine stimulation. WT (+/+) mice and mice with null mutations (−/−) of IL-18Rα or IFN-γ were exposed to CS or RA (CS−) for 2 weeks and then given 1 (FIGS. 5A, 5B) or 4 doses (FIG. 5C) of Poly(I:C) (50 μg) or vehicle control. BAL IL-18 (FIG. 5A), IL-12/23 p40 (FIG. 5B) and IFN-γ (FIG. 5C) levels were quantitated. The noted values represent the mean+SEM of evaluations in a minimum of 5 mice (*p<0.05, **p<0.01, ***p<0.001; ND=none detected).

FIG. 6, comprising FIGS. 6A through 6D, is a series of images demonstrating the roles of TLR-3 in the interaction of CS and Poly(I:C). WT (+/+) mice and mice with null mutations (−/−) of TLR-3 were exposed to CS or. RA (CS−) for 2 weeks and then given 1 or 4 doses of Poly(I:C) (50 μg) or vehicle control. BAL total cell recovery after a single dose of Poly(I:C) is illustrated in FIG. 6A. The levels of BAL IFN-γ (FIG. 6B), BAL total cell recovery (FIG. 6C), and alveolar remodeling (FIG. 6D) after 4 doses of Poly (I:C) were also evaluated. The noted values represent the mean+SEM of evaluations in a minimum of 5 mice (*p<0.05).

FIG. 7, comprising FIGS. 7A through 7C, is a series of images demonstrating the roles of MAVS in the interaction of CS and Poly(I:C). WT (+/+) mice and mice with null mutations (−/−) of MAVS were exposed to CS or RA (CS−) for 2 weeks and then given 4 doses of Poly(I:C) (50 μg) or vehicle control. BAL total cell recovery (FIG. 7A), IFN-γ production (FIG. 7B) and alveolar remodeling (FIG. 7C) were evaluated. The noted values represent the mean+SEM of evaluations in a minimum of 5 mice (*p<0.05, **p<0.01).

FIG. 8, comprising FIGS. 8A through 8E, is a series of images demonstrating the regulation and roles of PKR in the interaction of CS and Poly(I:C). Mice were exposed to CS or RA (CS−) for 2 weeks and then given 4 doses of Poly(I:C) (50 μg) (PolyI:C+) or vehicle control (PolyI:C−). FIG. 8A illustrates the effects of Poly(I:C) on PKR phosphorylation. The roles of IL-18Rα and IFN-γ in this activation are seen in comparisons of the levels of PKR phosphorylation in WT (+/+) mice and mice with null (−/−) mutations of IL-18Rα or IFN-γ (FIG. 8B). The role(s) of MAVS in this activation are seen in comparisons of the levels of PKR phosphorylation in WT (+/+) mice and mice with null (−/−) mutations of MAVS (FIG. 8C). The roles of PKR in Poly(I:C)-induced total (FIG. 8D) and differential (FIG. 8E) BAL cell recovery and emphysema (FIG. 8F) are also illustrated. The values in FIGS. 8D-8F represent the mean+SEM of evaluations in a minimum of 5 mice. FIGS. 8A-8C are representative of a minimum of 4 similar evaluations (*p<0.05, **p<0.01).

FIG. 9, comprising FIGS. 9A through 9F, is a series of images demonstrating the effects of CS and Poly(I:C) on cellular apoptosis. Mice were exposed to CS or RA (CS−) for 2 weeks and then given 4 doses of Poly(I:C) (50 μg) (PolyI:C+) or vehicle control (PolyI:C−). The, effects of these interventions on TUNEL staining can be seen in FIG. 9A. Double label IHC was used to define the percentage of pro-SP-C positive alveolar type II cells, CD31 positive endothelial cells and CCSP positive airway epithelial cells that were TUNEL staining respectively (FIG. 9B). The effects of CS and Poly (I:C) on caspase-3 activation, PARP accumulation and activation and eIF2α accumulation and activation were also assessed (FIG. 9C). The role of MAVS was assessed by comparing the levels of TUNEL staining, phosphorylation of eIF2α and caspase 3 activation in WT mice (+/+) and mice with null (−/−) mutations of MAVS (FIGS. 9D-9E). The roles of IL-18Rα, IFN-γ and PKR were assessed by comparing these responses in WT mice. (+/+) and mice with null (−/−) mutations of IL-18Rα, IFN-γ or PKR (FIGS. 9F and 9G). FIGS. 9C, 9E and 9G are representative of a minimum of 4 similar evaluations. The values in FIGS. 9A, 9B, 9D, and 9F represent the mean+SEM of evaluations in a minimum of 5 mice (*p<0.05, **p<0.01, ***p<0.001).

FIG. 10, comprising FIGS. 10A through 10I, is a series of Interactions of influenza virus and CS. Mice were exposed to CS or RA (CS−) for 2 weeks and then infected with influenza virus (virus+) or vehicle control (virus−). The effects of this intervention on total BAL cell recovery (FIG. 10A), differential cell recovery (9 days after infection) (FIG. 10B), tissue inflammation (magnification ×20) (FIG. 10C), the levels of BAL IL-18 (FIG. 10D), BAL IL-12/23p40 (FIG. 10E) and BAL IFN-γ (FIG. 10F) are compared in mice exposed to RA (open circles) and CS (solid circles). The effects of this intervention on caspase 3 activation, PARP cleavage and PKR and EIF-2α phosphorylation (FIG. 10G), cellular apoptosis (FIG. 10H), and alveolar remodeling (15 days after infection) (FIG. 10I) were also assessed. The values in FIGS. 10A, 10B, 10D-10F, 10H and 10I represent the mean+SEM of evaluations in a minimum of 5 mice. FIGS. 10C and 10G are representative of 4 similar evaluations (*p<0.05, **p<0.01, ***p<0.001).

FIG. 11, comprising FIGS. 11A through 11E, is a series of images demonstrating the roles of TLR-3, IL-18Rα and PKR in the inflammatory and remodeling effects of influenza virus and CS. Mice were exposed to CS or RA (CS−) for 2 weeks and then infected with influenza virus (virus+) or vehicle control (virus−). The effects of this intervention on total BAL cell recovery 9 days after viral inoculation is illustrated in FIG. 11A. Lung volume (FIG. 11B), mean chord length (FIG. 11C), TUNEL staining (FIG. 11D) and alveolar histology (FIG. 11E) were assessed 15 days after viral inoculation. FIGS. 11A-11D represent the mean+SEM of evaluations in a minimum of 5 mice. FIG. 11E is representative of 4 similar evaluations.

### DETAILED DESCRIPTION OF THE INVENTION

The present invention is based upon the discovery that cigarette smoke is a potent and selective enhancer of viral induced inflammation, alveolar remodeling, and cell apoptosis in lung. Accordingly, the present invention encompasses compositions and methods for the treatment and prevention of episodic exacerbations of COPD that are often caused by viral infection.

### Definitions:

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques or modifications thereof, are used for chemical syntheses and chemical analyses.

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Alveolar remodeling” refers to one or more changes in alveolar cells observed in various pulmonary diseases and disorders, including, but not limited to, pulmonary epithelial cell DNA destruction, apoptosis, pathological accumulation of collagen, alveolar honeycombing, and alveolar septal destruction.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator,” as the term is used herein, is meant any device including, but not limited to, a hypodermic syringe, a pipette, and the like, for administering the compounds and compositions of the invention.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

A “putative at-risk individual” is a mammal, preferably a human, who is thought to be at risk of developing COPD.

The term “BAL,” as used herein, refers to bronchoalveolar lavage fluid.

The term “chronic obstructive pulmonary disease,” or COPD, is used herein to refer to two lung diseases, chronic bronchitis and emphysema, that are characterized by obstruction to airflow that interferes with normal breathing. Both of these conditions frequently co-exist.

The term “emphysema” is a major subset of the clinical entity known as COPD and is characterized by specific pathological changes in lung tissue over time. One hallmark of emphysema is the gradual, progressive, and irreversible destruction of the distal lung parenchyma leading to the destruction alveoli. Alveolar destruction leads to enlarged airspaces in the lung and consequently a reduced ability to transfer oxygen to the bloodstream. Emphysema is also characterized by a loss of elasticity in the lung making it difficult to maintain open airways. Both of these changes produce the clinical sequelae of emphysema comprising shortness of breath and difficulty exhaling, respectively.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease, or disorder, the frequency with which such a symptom is experienced by a patient, or both, are reduced.

The terms “dysregulated” or “dysregulation,” as used herein, refer to an impairment in a biological process which in turn may lead to deleterious physiological sequelae, or abnormal expression of a gene, nucleic acid, protein, peptide, or other biological molecule. In the case where expression of a gene, nucleic acid, protein, peptide, or other biological molecule is dysregulated, the gene, nucleic acid,protein, peptide, or other biological molecule is expressed, processed, or maintained at levels that are outside what is considered the normal range for that of that gene, nucleic acid, protein, peptide, or other biological molecule as determined by a skilled artisan. Dysregulation of a gene, nucleic acid, protein, peptide, or other biological molecule in a mammal may be determined by measuring the level of a gene, nucleic acid, protein, peptide, or other biological molecule in the mammal and comparing the level measured in that mammal to level measured in a matched population known not to be experiencing dysregulation of that gene, nucleic acid, protein, peptide, or other biological molecule dysregulated. Alternatively, the level may be compared to one measured in the same individual at a different time.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “recombinant DNA” as used herein is defined as DNA produced by joining pieces of DNA from different sources.

The term “recombinant polypeptide” as used herein is defined as a polypeptide produced by using recombinant DNA methods.

“Ribozymes” as used herein are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053).

By the term “specifically binds,” as used herein, is meant a molecule, such as an antibody, which recognizes and binds to another molecule or feature, but does not substantially recognize or bind other molecules or features in a sample.

As used herein, the term “transdominant negative mutant gene” refers to a gene encoding a protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The phrase “inhibitor,” including an “IL-18 inhibitor,” an “IL-18Rα inhibitor,” an “IFNγ inhibitor,” and a “double stranded RNA-dependent protein kinase (PKR) inhibitor,” as used herein, refers to a composition or compound that inhibits the activity, function, interaction, binding, stability, or expression of a molecule either directly or indirectly, using any method known to the skilled artisan. An inhibitor may be any type of compound, including but not limited to, a polypeptide, a nucleic acid, an aptamer, a peptidometic, and a small molecule.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

It is understood that any and all whole or partial integers between any ranges set forth herein are included herein.

### Description:

The present invention is based in part on the discovery that cigarette smoke (CS) selectively enhances viral pathogen-associated molecular pattern (PAMP) induced lung inflammation, alveolar remodeling, and cell apoptosis which lead to the development or exacerbation of COPD. The interaction of CS and viral PAMP is associated with the early induction of IL-18 and the later induction of IL-12/23 and IFN-γ mediated by IL-18Rα, IFN-γ, and double stranded RNA-dependent kinase (PKR). The data disclosed elsewhere herein provide, for the first time, a novel mechanistic based approach to treating a mammal with COPD as well as treating and preventing episodic COPD exacerbation due to viral infection.

Accordingly, the present invention encompasses compositions and methods useful in treating an individual diagnosed with COPD. Treating an individual diagnosed with COPD encompasses a method of inhibiting the progression of COPD in an individual diagnosed with COPD.

By “inhibiting the progression of COPD” is intended to mean that the progressive histological and morphometric changes associated with the clinical sequelae of COPD, including inflammation, alveolar remodeling, and lung cell death, are halted, prevented, or attenuated.

It will be appreciated that the methods of the present invention may also be practiced on an individual at risk of developing COPD whereby an individual identified as being at risk of developing COPD may be prevented from developing or experiencing inflammation, alveolar remodeling, and lung cell death that would subsequently lead to a clinical manifestation of COPD.

In another embodiment, the methods of the present invention comprise inhibiting inflammation, alveolar remodeling, and/or apoptosis in the lung of a mammal.

The methods of the invention comprise administering a therapeutically effective amount of an IL-18 inhibitor to an individual with COPD, or an individual at risk of developing COPD, where the inhibitor reduces, or prevents, halts, or attenuates inflammation, alveolar remodeling, or lung cell apoptosis.

The methods of the invention also comprise administering a therapeutically effective amount of an IL-18Rα inhibitor to an individual with COPD or an individual at risk of developing COPD where the inhibitor reduces, or prevents, halts, or attenuates inflammation, alveolar remodeling, or lung cell apoptosis.

The methods of the invention further comprise administering a therapeutically effective amount of an IFN-γ inhibitor to an individual with COPD or an individual at risk of developing COPD where the inhibitor reduces, or prevents, halts, or attenuates inflammation, alveolar remodeling, or lung cell apoptosis.

The methods of the invention also comprise administering a therapeutically effective amount of a PKR inhibitor to an individual with COPD or an individual at risk of developing COPD where the inhibitor reduces, or prevents, halts, or attenuates inflammation, alveolar remodeling, or lung cell apoptosis.

The methods of the present invention may be practiced on any individual diagnosed with, or at risk of developing COPD. Preferably the individual is a human. An individual may have COPD, or be at risk of developing COPD because of a history of smoking, exposure to environmental pollutants, toxins, infectious agents, or other compounds that may induce inflammation, alveolar remodeling, and/or cell apoptosis in lung. An individual who smokes, or who has been diagnosed with COPD and who has a known exposure to, or risk of exposure to a infectious virus, such as influenza A, would also be a candidate for treatment according to the methods of the present invention.

Inhibiting IL-18, IL-18-Rα, IFN-γ, and/or PKR activity can be accomplished using any method known to the skilled artisan. Examples of methods to inhibit IL-18, IL-18-Rα, IFN-γ, and/or PKR activity include, but are not limited to decreasing expression of an endogenous IL-18, IL-18-Rα, IFN-γ, and/or PKR gene, decreasing expression of IL-18, IL-18-Rα, IFN-γ, and/or PKR mRNA, and inhibiting activity of IL-18, IL-18-Rα, IFN-γ, and/or PKR protein. An IL-18, IL-18-Rα, IFN-γ, and/or PKR inhibitor may therefore be a compound or composition that decreases expression of a IL-18, IL-18-Rα, IFN-γ, and/or PKR gene, a compound or composition that decreases IL-18, IL-18-Rα, IFN-γ, and/or PKR mRNA half-life, stability and/or expression, or a compound or composition that inhibits IL-18, IL-18-Rα, IFN-γ, and/or PKR protein function. A IL-18, IL-18-Rα, IFN-γ, and/or PKR inhibitor may be any type of compound, including but not limited to, an antibody, a polypeptide, a nucleic acid, an aptamer, a peptidometic, and a small molecule, or combinations thereof.

IL-18, IL-18-Rα, IFN-γ, and/or PKR inhibition may be accomplished either directly or indirectly. For example, IL-18, IL-18-Rα, IFN-γ, and/or PKR may be directly inhibited by compounds or compositions that directly interact with IL-18, IL-18-Rα, IFN-γ, and/or PKR protein, such as antibodies or proteinase inhibitors. Alternatively, IL-18, IL-18-Rα, IFN-γ, and/or PKR may be inhibited indirectly by compounds or compositions that inhibit IL-18, IL-18-Rα, IFN-γ, and/or PKR downstream effectors, or upstream regulators which up-regulate IL-18, IL-18-Rα, IFN-γ, and/or PKR expression.

Decreasing expression of an endogenous IL-18, IL-18-Rα, IFN-γ, and/or PKR gene includes providing a specific inhibitor of IL-18, IL-18-Rα, IFN-γ, and/or PKR gene expression. Decreasing expression of IL-18, IL-18-Rα, IFN-γ, and/or PKR mRNA or IL-18, IL-18-Rα, IFN-γ, and/or PKR protein includes decreasing the half-life or stability of IL-18, IL-18-Rα, IFN-γ, and/or PKR mRNA or decreasing expression of IL-18, IL-18-Rα, IFN-γ, and/or PKR mRNA. Methods of decreasing expression of IL-18, IL-18-Rα, IFN-γ, and/or PKR include, but are not limited to, methods that use an siRNA, a microRNA, an antibody, a proteinase, an antisense nucleic acid, a ribozyme, an expression vector encoding a transdominant negative mutant, a peptide, a small molecule, other specific inhibitors of IL-18, IL-18-Rα, IFN-γ, and/or PKR gene, mRNA, and protein expression, and combinations thereof.

The present invention should in no way be construed to be limited to the inhibitors or activators described herein, but rather should be construed to encompass any activator or inhibitor of the IL-18, IL-18-Rα, IFN-γ, and/or PKR in lung, both known and unknown, that promotes lung structural integrity or prevents, attenuates, or halts the development of pathophysological changes in lung associated with COPD.

### I. Compositions

A. IL-18, IL-18-Rα, IFN-γ, and/or PKR Inhibitors

1. Antibodies

In one embodiment of the invention, the IL-18, IL-18-Rα, IFN-γ, and/or PKR inhibitor is an antibody. It will be appreciated by one skilled in the art that an antibody comprises any immunoglobulin molecule, whether derived from natural sources or from recombinant sources, which is able to specifically bind to an epitope present on a target molecule. In the present invention, the target molecule may be IL-18, IL-18-Rα, IFN-γ, and/or PKR, or fragments thereof. In one aspect of the invention, IL-18, IL-18-Rα, IFN-γ, and/or PKR is directly inhibited by an antibody that specifically binds to an epitope on IL-18, IL-18-Rα, IFN-γ, and/or PKR. In another aspect of the invention, the effects of IL-18, IL-18-Rα, IFN-γ, and/or PKR are blocked by an antibody that specifically binds to an epitope on a downstream effector such as extracellular matrix (ECM) proteins, proteases, anti-proteases, transcription factors, fibrogenetic cytokines, or apoptosis regulators. In still another aspect of the invention, the effects of IL-18, IL-18-Rα, IFN-γ, and/or PKR are blocked by an antibody that binds to an epitope of an upstream regulator of IL-18, IL-18-Rα, IFN-γ, and/or PKR.

When the IL-18, IL-18-Rα, IFN-γ, and/or PKR inhibitor used in the compositions and methods of the invention is a polyclonal antibody (IgG), the antibody is generated by inoculating a suitable animal with a peptide comprising full length IL-18, IL-18-Rα, IFN-γ, and/or PKR protein, or a fragment thereof, an upstream regulator, or fragments thereof. These polypeptides, or fragments thereof, may be obtained by any methods known in the art, including chemical synthesis and biological synthesis, as described elsewhere herein. In this regard, exemplary human sequences are shown in table 1. Antibodies produced in the inoculated animal which specifically bind to IL-18, IL-18-Rα, IFN-γ, and/or PKR, or fragments thereof, are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1998, In: Antibodies, A Laboratory Manual, Cold Spring Harbor, N.Y.).

Monoclonal antibodies directed against a full length IL-18, IL-18-Rα, IFN-γ, and/or PKR, or fragment thereof, may be prepared using any well known monoclonal antibody preparation procedures, such as those described, for example, in Harlow et al. (1998, In: Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein.

When the antibody used in the methods of the invention is a biologically active antibody fragment or a synthetic antibody corresponding to antibody to a full length IL-18, IL-18-Rα, IFN-γ, and/or PKR, or fragments thereof, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with an epitope present on a target molecule. These antibodies are capable of binding to the target molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen, et al. (U.S. Pat. No. 6,180,370), Wright et al., (supra) and in the references cited therein, or in Gu et al. (1997, Thrombosis and Hematocyst 77(4):755-759), or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to humanized immunoglobulin coding sequences, including naturally-associated or heterologous promoter regions. The expression control sequences can be eukaryotic promoter systems in vectors capable of transforming or transfecting eukaryotic host cells, or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to the target molecule. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1998, Antibodies: A Laboratory Manual, Cold Spring Harbor, N.Y.).

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as IL-18, IL-18-Rα, IFN-γ, and/or PKR inhibitors in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J. Mol. Biol. 248:97-105).

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used to practice the method of the invention, or to prepare a pharmaceutical composition useful in practicing the method of the invention.

The antibodies of the present invention can be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g, Current Protocols in Molecular Biology, (Ausubel et al., eds.), Greene Publishing Associates and Wiley-Interscience, New York (2002)). Exemplary immunoassays are described briefly below (but are not intended to be in any way limiting).

2. Inhibitors of IL-18, IL-18-Rα, IFN-γ, and/or PKR Gene and mRNA Expression

a. Antisense Nucleic Acids

In one embodiment of the invention, an antisense nucleic acid sequence which is expressed by a plasmid vector is used to inhibit IL-18, IL-18-Rα, IFN-γ, and/or PKR expression. The antisense expressing vector is used to transfect a mammalian cell or the mammal itself, thereby causing reduced endogenous expression of IL-18, IL-18-Rα, IFN-γ, and/or PKR, or a regulator thereof, such as Nox3.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

b. Ribozymes

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a mariner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit IL-18, IL-18-Rα, IFN-γ, and/or PKR expression. Ribozymes useful for inhibiting the expression of a target molecule may be designed by incorporating target sequences into the basic ribozyme structure which are complementary, for example, to the mRNA sequence of IL-18, IL-18-Rα, IFN-γ, and/or PKR of the present invention. Ribozymes targeting IL-18, IL-18-Rα, IFN-γ, and/or PKR, or an upstream regulator thereof, may be synthesized using commercially available reagents (Applied Biosystems, Inc., Foster City, Calif.) or they may be genetically expressed from DNA encoding them.

c. siRNA

In one embodiment, siRNA is used to decrease the level of IL-18, IL-18-Rα, IFN-γ, and/or PKR protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14(7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of IL-18, IL-18-Rα, IFN-γ, and/or PKR protein using RNAi technology.

i. Modification of siRNA

Following the generation of the siRNA polynucleotide of the present invention, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987 Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide of the invention may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

ii. Vectors

In other related aspects, the invention includes an isolated nucleic acid encoding an inhibitor, wherein the inhibitor such as an siRNA, inhibits IL-18, IL-18-Rα, IFN-γ, and/or PKR, or a regulator thereof, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York). In another aspect of the invention, IL-18, IL-18-Rα, IFN-γ, and/or PKR, or a regulator thereof, can be inhibited by way of inactivating and/or sequestering IL-18, IL-18-Rα, IFN-γ, and/or PKR, or a regulator thereof. As such, inhibiting the effects of IL-18, IL-18-Rα, IFN-γ, and/or PKR can be accomplished by using a transdominant negative mutant.

In another aspect, the invention includes a vector comprising an siRNA polynucleotide. Preferably, the siRNA polynucleotide is capable of inhibiting the expression of a target polypeptide, wherein the target polypeptide is selected from the group consisting of IL-18, IL-18-Rα, IFN-γ, and/or PKR, or regulators thereof. The incorporation of a desired polynucleotide into a vector and the choice of vectors is well-known in the art as described in, for example, Sambrook et al., supra, and Ausubel et al., supra.

The siRNA polynucleotide can be cloned into a number of types of vectors. However, the present invention should not be construed to be limited to any particular vector. Instead, the present invention should be construed to encompass a wide plethora of vectors which are readily available and/or well-known in the art. For example, an siRNA polynucleotide of the invention can be cloned into a vector including, but not limited to a plasmid, a phagemid, a phage derivative, an animal viruses, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the siRNA, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 by upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 by apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid, sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the siRNA, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

3. Peptides

When the IL-18, IL-18-Rα, IFN-γ, and/or PKR inhibitor is a peptide, the peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the a-amino protecting group, and the FMOC method, which utilizes 9-fluorenylmethyloxcarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a IL-18, IL-18-Rα, IFN-γ, and/or PKR inhibitor in accordance with the invention, a peptide is purified to remove contaminants: Any one of a number of a conventional purification procedures may be used to attain the required level of purity including, for example, reversed-phase high-pressure liquid chromatography (HPLC) using an alkylated silica column such as C₄-, C₈- or C₁₈- silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

4. Small Molecules

When the IL-18, IL-18-Rα, IFN-γ, and/or PKR inhibitor is a small molecule, a small molecule activator may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making said libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

5. Identifying and Testing Candidate IL-18, IL-18-Rα, IFN-γ, and/or PKR Inhibitors

IL-18, IL-18-Rα, IFN-γ, and/or PKR inhibitors comprising inhibitors of gene expression, mRNA stability and expression, protein activity, function and expression of IL-18, IL-18-Rα, IFN-γ, and/or PKR, upstream regulators, and downstream effectors can be identified by screening test compounds. For instance, inhibitors of endogenous IL-18, IL-18-Rα, IFN-γ, and/or PKR gene expression or of IL-18, IL-18-Rα, IFN-γ, and/or PKR mRNA expression can be identified by screening test compounds for their capacity to reduce or preclude IL-18, IL-18-Rα, IFN-γ, and/or PKR gene expression or IL-18, IL-18-Rα, IFN-γ, and/or PKR mRNA expression in a cell, preferably a pulmonary endothelial cell. The IL-18, IL-18-Rα, IFN-γ, and/or PKR coding sequence in such screening assays may include an in-frame fusion of a tag to the IL-18, IL-18-Rα, IFN-γ, and/or PKR coding sequence. Such tags enable monitoring of IL-18, IL-18-Rα, IFN-γ, and/or PKR expression by antibody detection of the tags or spectral methods of detection (e.g., fluorescence or luminescence).

Test compounds for use in such screening methods can be small molecules, nucleic acids including aptamers, peptides, peptidomimetics and other drugs. Peptide fragments of IL-18, IL-18-Rα, IFN-γ, and/or PKR are contemplated that can competitively inhibit the binding of IL-18, IL-18-Rα, IFN-γ, and/or PKR to a downstream effector molecule, thereby inhibiting IL-18, IL-18-Rα, IFN-γ, and/or PKR activity.

The test compounds of the present invention can be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145). Inhibitors of IL-18, IL-18-Rα, IFN-γ, and/or PKR expression may be useful in therapeutic applications, or serve as lead drugs in the development of therapeutics. Synthetic techniques may be used to produce compounds, such as: chemical and enzymatic production of small molecules, peptides, nucleic acids, antibodies, and other therapeutic compositions useful in the practice of the methods of the invention. Other techniques may be used which are not described herein, but are known to those of skill in the art.

### II. Methods

The present invention provides a method of treating COPD in a mammal. In one embodiment, the present invention provides a method of preventing a mammal at-risk of developing COPD from developing pathophysiological changes and clinical sequelae associated with COPD.

The method of the invention comprises administering a therapeutically effective amount of at least one IL-18, IL-18-Rα, IFN-γ, and/or PKR inhibitor, or a combination thereof, to a mammal wherein a IL-18, IL-18-Rα, IFN-γ, and/or PKR inhibitor, or combination thereof prevents, attenuates, or halts the pathophysiological changes associated with COPD in lung, including but not limited to decreased inflammation, alveolar remodeling, and cellular apoptosis.

In another embodiment, the method of the invention comprises administering a therapeutically effective amount of at least one IL-18, IL-18-Rα, IFN-γ, and/or PKR inhibitor, or a combination thereof, to a mammal wherein a IL-18, IL-18-Rα, IFN-γ, and/or PKR inhibitor, or combination thereof is used to treat a mammal diagnosed with a disease or disorder inflammation, alveolar remodeling, and cellular apoptosis in lung is a component of the disease or disorder.

In still another embodiment, the method of the invention comprises administering a therapeutically effective amount of at least one IL-18, IL-18-Rα, IFN-γ, and/or PKR inhibitor, or a combination thereof, is used to treat COPD.

In still another embodiment, the method of the invention comprises administering a therapeutically effective amount of at least one IL-18, IL-18-Rα, IFN-γ, and/or PKR inhibitor, or a combination thereof, is used to prevent the development of COPD in an individual at-risk of developing COPD.

In yet another embodiment, the method of the present invention comprises administering at least one IL-18, IL-18-Rα, IFN-γ, and/or PKR inhibitor, or a combination thereof to treat, prevent, reduce or attenuate inflammation, alveolar remodeling, apoptosis, or a combination thereof.

The subject may be diagnosed with a disease or disorder wherein the disease or disorder has a dysregulation of the IL-18, IL-18-Rα, IFN-γ, and/or PKR expression in lung as part of the disease's clinical features. Alternatively, the subject may be at-risk of developing a disease or disorder wherein the disease or disorder has a dysregulation of IL-18, IL-18-Rα, IFN-γ, and/or PKR expression in lung as part of the disease's clinical features. Examples of a disease or disorder which may be treated using the methods of the present invention include but are not limited to chronic obstructive pulmonary disease (COPD) and emphysema. In a preferred embodiment the subject is a mammal. In a more preferred embodiment the subject is a human.

Methods of prophylaxis (i.e., prevention or decreased risk of disease), as well as reduction in the frequency or severity of symptoms associated with COPD or any related disease or disorder, are encompassed by the present invention.

The method of the invention comprises administering a therapeutically effective amount of at least one IL-18, IL-18-Rα, IFN-γ, and/or PKR inhibitor, or a combination thereof, to a mammal wherein a composition of the present invention comprising a IL-18, IL-18-Rα, IFN-γ, and/or PKR inhibitor, or a combination thereof is used either alone or in combination with other therapeutic agents to treat a subject. A IL-18, IL-18-Rα, IFN-γ, and/or PKR inhibitor, or a combination thereof may be administered either, before, during, after, or throughout the administration of said therapeutic agent. The compositions and methods of the present invention can be used in combination with other treatment regimens, including virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone), steroids (including prednisone and dexamethasone), and antidepressants (including gabapentin, amitriptyline, imipramine, doxepin) antihistamines, antitussives, muscle relaxants, bronchodilators, beta-agonists, anticholinergics, corticosteroids, mast cell stabilizers, leukotriene modifiers, methylxanthines, as well as combination therapies, and the like. The invention can also be used in combination with other treatment modalities, such as chemotherapy, cryotherapy, hyperthermia, radiation therapy, and the like.

C. Methods of Delivering a IL-18, IL-18-Rα, IFN-γ, and/or PKR Inhibitor to a Cell

The present invention comprises a method for treating or preventing inflammation, alveolar remodeling and/or cell apoptosis in the as well as the development of COPD in a mammal, said method comprising administering a therapeutic amount of an IL-18, IL-18-Rα, IFN-γ, and/or PKR inhibitor, or a combination thereof to said mammal. In particular, the invention includes a method for attenuating inflammation, lung cell apoptosis, and alveolar remodelling, all of which are features of COPD.

Isolated nucleic acid-based IL-18, IL-18-Rα, IFN-γ, and/or PKR inhibitors can be delivered to a cell in vitro or in vivo using viral vectors comprising one or more isolated IL-18, IL-18-Rα, IFN-γ, and/or PKR inhibitor sequences. Generally, the nucleic acid sequence has been incorporated into the genome of the viral vector. The viral vector comprising an isolated IL-18, IL-18-Rα, IFN-γ, and/or PKR inhibitor nucleic acid described herein can be contacted with a cell in vitro or in vivo and infection can occur. The cell can then be used experimentally to study, for example, the effect of an isolated IL-18, IL-18-Rα, IFN-γ, and/or PKR inhibitor in vitro, or the cells can be implanted into a subject for therapeutic use. The cell can be migratory, such as a hematopoietic cell, or non-migratory. The cell can be present in a biological sample obtained from the subject (e.g., blood, bone marrow, tissue, fluids, organs, etc.) and used in the treatment of disease, or can be obtained from cell culture.

After contact with the viral vector comprising an isolated IL-18, IL-18-Rα, IFN-γ, and/or PKR inhibitor nucleic acid sequence, the sample can be returned to the subject or re-administered to a culture of subject cells according to methods known to those practiced in the art. In the case of delivery to a subject or experimental animal model (e.g., rat, mouse, monkey, chimpanzee), such a treatment procedure is sometimes referred to as ex vivo treatment or therapy. Frequently, the cell is removed from the subject or animal and returned to the subject or animal once contacted with the viral vector comprising the isolated inhibitor nucleic acid of the present invention. Ex vivo gene therapy has been described; for example, in Kasid et al., Proc. Natl. Acad. Sci. USA 87:473 (1990); Rosenberg et al, New Engl. J Med. 323:570 (1990); Williams et al., Nature 310476 (1984); Dick et al., Cell 42:71 (1985); Keller et al., Nature 318:149 (1985) and Anderson et al., U.S. Pat. No. 5,399,346 (1994).

Where a cell is contacted in vitro, the cell incorporating the viral vector comprising an isolated IL-18, IL-18-Rα, IFN-γ, and/or PKR inhibitor nucleic acid can be implanted into a subject or experimental animal model for delivery or used in in vitro experimentation to study cellular events mediated by IL-18, IL-18-Rα, IFN-γ, and/or PKR inhibitor activity.

Various viral vectors can be used to introduce an isolated IL-18, IL-18-Rα, IFN-γ, and/or PKR inhibitor nucleic acid into mammalian cells. Viral vectors include retrovirus, adenovirus, parvovirus (e.g., adeno-associated viruses), coronavirus, negative-strand RNA viruses such as orthomyxovirus (e.g., influenza virus), rhabdovirus (e.g., rabies and vesicular stomatitis virus), paramyxovirus (e.g. measles and Sendai), positive-strand RNA viruses such as picornavirus and alphavirus, and double stranded DNA viruses including adenovirus, herpesvirus (e.g., herpes simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus, lentiviruses and baculoviruses.

In addition, an engineered viral vector can be used to deliver an isolated IL-18, IL-18-Rα, IFN-γ, and/or PKR inhibitor nucleic acid of the present invention. These vectors provide a means to introduce nucleic acids into cycling and quiescent cells, and have been modified to reduce cytotoxicity and to improve genetic stability. The preparation and use of engineered Herpes simplex virus type 1 (Krisky et al., 1997, Gene Therapy 4:1120-1125), adenoviral (Amalfitanl et al., 1998, Journal of Virology 72:926-933) attenuated lentiviral (Zufferey et al., 1997, Nature Biotechnology 15:871-875) and adenoviral/retroviral chimeric (Feng et al., 1997, Nature Biotechnology 15:866-870) vectors are known to the skilled artisan. In addition to delivery through the use of vectors, an isolated IL-18, IL-18-Rα, IFN-γ, and/or PKR inhibitor nucleic acid can be delivered to cells without vectors, e.g. as “naked” nucleic acid delivery using methods known to those of skill in the art. See, for example, U.S. Pat. Nos. 5,350,674 and 5,585,362.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Various forms of an isolated IL-18, IL-18-Rα, IFN-γ, and/or PKR inhibitor nucleic acid, as described herein, can be administered or delivered to a mammalian cell (e.g., by virus, direct injection, or liposomes, or by any other suitable methods known in the art or later developed). The methods of delivery can be modified to target certain cells, and in particular, cell surface receptor molecules. As an example, the use of cationic lipids as a carrier for nucleic acid constructs provides an efficient means of delivering the isolated TLR agonist nucleic acid of the present invention.

Various formulations of cationic lipids have been used to deliver nucleic acids to cells (WO 91/17424; WO 91/16024; U.S. Pat. Nos. 4,897,355; 4,946,787; 5,049,386; and 5,208,036). Cationic lipids have also been used to introduce foreign polynucleotides into frog and rat cells in vivo (Holt et al., Neuron 4:203-214 (1990); Hazinski et al., Am. J. Respr. Cell. Mol. Biol. 4:206-209 (1991)). Therefore, cationic lipids may be used, generally, as pharmaceutical carriers to provide biologically active substances (for example, see WO 91/17424; WO 91/16024; and WO 93/03709). Thus, cationic liposomes can provide an efficient carrier for the introduction of polynucleotides into a cell.

Further, liposomes can be used as carriers to deliver a nucleic acid to a cell, tissue or organ. Liposomes comprising neutral or anionic lipids do not generally fuse with the target cell surface, but are taken up phagocytically, and the polynucleotides are subsequently subjected to the degradative enzymes of the lysosomal compartment (Straubinger et al., 1983, Methods Enzymol. 101:512-527; Mannino et al., 1988, Biotechniques 6:682-690). However, as demonstrated by the data disclosed herein, an isolated snRNA of the present invention is a stable nucleic acid, and thus, may not be susceptible to degradative enzymes. Further, despite the fact that the aqueous space of typical liposomes may be too small to accommodate large macromolecules, the isolated IL-18, IL-18-Rα, IFN-γ, and/or PKR inhibitor nucleic acid of the present invention is relatively small, and therefore, liposomes are a suitable delivery vehicle for the present invention. Methods of delivering a nucleic acid to a cell, tissue or organism, including liposome-mediated delivery, are known in the art and are described in, for example, Feigner (Gene Transfer and Expression Protocols Vol. 7, Murray, E. J. Ed., Humana Press, New Jersey, (1991)).

In other related aspects, the invention includes an isolated IL-18, IL-18-Rα, IFN-γ, and/or PKR inhibitor nucleic acid operably linked to a nucleic acid comprising a promoter/regulatory sequence such that the nucleic acid is preferably capable of delivering an isolated IL-18, IL-18-Rα, IFN-γ, and/or PKR inhibitor nucleic acid. Thus, the invention encompasses expression vectors and methods for the introduction of an isolated IL-18, IL-18-Rα, IFN-γ, and/or PKR inhibitor nucleic acid into or to cells.

Such delivery can be accomplished by generating a plasmid, viral, or other type of vector comprising an isolated IL-18, IL-18-Rα, IFN-γ, and/or PKR inhibitor nucleic acid operably linked to a promoter/regulatory sequence which serves to introduce the IL-18, IL-18-Rα, IFN-γ, and/or PKR inhibitor into cells in which the vector is introduced. Many promoter/regulatory sequences useful for the methods of the present invention are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of an isolated IL-18, IL-18-Rα, IFN-γ, and/or PKR inhibitor nucleic acid may be accomplished by placing an isolated IL-18, IL-18-Rα, IFN-γ, and/or PKR inhibitor nucleic acid, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Selection of any particular plasmid vector or other vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York) and elsewhere herein.

### III. Pharmaceutical Compositions and Therapies

Administration of a IL-18, IL-18-Rα, IFN-γ, and/or PKR inhibitor comprising one or more peptides, small molecules, antisense nucleic acids, or antibodies of the invention in a method of treatment can be achieved in a number of different ways, using methods known in the art. Such methods include, but are not limited to, providing exogenous peptide inhibitor, small molecule, or an antibody to a subject or expressing a recombinant peptide inhibitor, small molecule, soluble receptor, or an antibody expression cassette.

The therapeutic and prophylactic methods of the invention thus encompass the use of pharmaceutical compositions comprising IL-18, IL-18-Rα, IFN-γ, and/or PKR inhibitor peptide, fusion protein, small molecule, or antibody of the invention and/or an isolated nucleic acid encoding a IL-18, IL-18-Rα, IFN-γ, and/or PKR inhibitory peptide, fusion protein small molecule, or antibody of the invention to practice the methods of the invention. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 μg to about 10 mg per kilogram of body weight of the animal. More preferably, the dosage will vary from about 3 μg to about 1 mg per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutkally acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agent, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Kits

The invention also includes a kit comprising a IL-18, IL-18-Rα, IFN-γ, and/or PKR inhibitor, or a combination thereof, of the invention and an instructional material which describes, for instance, administering the IL-18, IL-18-Rα, IFN-γ, and/or PKR inhibitor, or a combination thereof to a subject as a prophylactic or therapeutic treatment or a non-treatment use as described elsewhere herein. In an embodiment, this kit further comprises a (preferably sterile) pharmaceutically acceptable carrier suitable for dissolving or suspending the therapeutic composition, comprising a IL-18, IL-18-Rα, IFN-γ, and/or PKR inhibitor, or a combination thereof of the invention, for instance, prior to administering the molecule to a subject. Optionally, the kit comprises an applicator for administering the inhibitor. In one embodiment of the invention, the applicator is designed for pulmonary administration of the IL-18, IL-18-Rα, IFN-γ, and/or PKR inhibitor, or combination thereof. In another embodiment, the kit comprises an antibody that specifically binds an epitope on IL-18, IL-18-Rα, IFN-γ, and/or PKR inhibitor, or a combination thereof. Preferably, the antibody recognizes a human IL-18, IL-18-Rα, IFN-γ, and/or PKR.

A kit providing a nucleic acid encoding a peptide or antibody of the invention and an instructional material is also provided.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### CS Exposure

Mice were exposed to room air (RA) or the smoke from nonfiltered standard research cigarettes (2R4, University of Kentucky) (CS) using the smoking apparatus described by Hautamaki et al., 1997, Science 277:2002-2004. Bronchoalveolar lavage fluid (BAL) and TUNEL evaluations were undertaken as described below. After 4 weeks, the mice were anesthetized and sacrificed, and the trachea was cannulated. After ligation of the right main bronchus, the left lung was inflated with 0.5% low temperature-melting agarose in 10% PBS-buffered formalin at a constant pressure of 25 cm. This allowed for homogenous expansion of lung. The lungs were then fixed in 10% PBS-buffered formalin for 24 hours, sectioned, and evaluated using histologic, immunohistologic, and morphologic methods as described below.

### BAL and Quantification of IL-18

Mice were euthanized, the trachea was isolated by blunt dissection, and tubing was secured in the airway. Three volumes of 0.6 ml of PBS were then instilled and gently aspirated and pooled. Each BAL fluid sample was centrifuged, and the supernatants were stored in −70° C. until used. Lung lysates were also prepared. The levels of IL-18 were determined using a commercial ELISA kit (R&D Systems) as per the manufacturer's instructions.

### Histologic Analysis

Animals were anesthetized, a median sternotomy was performed and right heart perfusion was accomplished with calcium- and magnesium-free PBS to clear the pulmonary intravascular space. The lungs were then fixed to pressure (25 cm) with neutral-buffered 10% formalin, fixed overnight in 10% formalin, embedded in paraffin, sectioned, and stained. H&E stains were performed in the Research Histology Laboratory of the Department of Pathology at Yale University School of Medicine.

mRNA Analysis

mRNA levels were assessed using real-time RT-PCR as previously described by our laboratory (Ma, et al., 2005, J. Clin. Invest. 115:3460-3472; Wang et al., 2000, J. Exp. Med. 192:1587-1600; Zheng, et al., 2000, J. CLin. Invest. 106:1081-1093). In these assays, gene-specific primers were used to amplify selected regions of each target moiety.

### Immunohistochemistry (IHC)

IHC was undertaken as described previously (Ma, et al., 2005, J. Clin. Invest. 115:3460-3472; Wang et al., 2000, J. Exp. Med 192:1587-1600; Zheng, et al., 2000, J. CLin. Invest. 106:1081-1093). Primary antiserum against murine IL-18 (Santa Cruz Biotechnology) was used.

### Chemokine Measurements

The levels of selected chemokines in BAL were evaluated by ELISA using commercial assays (R&D Systems) as described by the manufacturer.

Lung Volume, Morphometric, and Compliance Assessment

Lung volume, alveolar size, and lung compliance were assessed via volume displacement and morphometric chord length assessments as described previously ((Ma, et al., 2005, J. Clin. Invest. 115:3460-3472; Wang et al., 2000, J. Exp. Med. 192:1587-1600; Zheng, et al., 2000, J. CLin. Invest. 106:1081-1093)).

### TUNEL Evaluations

End labeling of exposed 3′-OH ends of DNA fragments in paraffin-embedded tissue was undertaken with the TUNEL in situ cell death detection kit AP (Roche Diagnostics) using the instructions provided by the manufacturer. Staining specificity was assessed by comparing the signal that was seen when terminal transferase was included and excluded from the reaction. After staining, a minimum of 20 fields of alveoli were randomly chosen, and 500 nuclei were counted per lung. The labeled cells were expressed as a percentage of total nuclei.

### Statistics

Normally distributed data are expressed as mean±SEM and were assessed for significance by Student's t test or ANOVA as appropriate. Data that were not normally distributed are expressed as media with interquartile ranges and were assessed for significance using the Mann-Whitney U test or the Kruskal-Wallis test with Dunn's posttest for multiple comparisons as appropriate. Statistical analysis was performed using Stata version 9.0 (StataCorp), Deltagraph (RockWare), and Prism version 4 (GraphPad). Statistical significance was defined at a level of p≦0.05.

The results of the experiments presented in this Example are now described.

### Example 1

### Cigarette Smoke (CS) and Poly(I:C) Regulate Lung Inflammation, Induce Emphysema, and Cell Apoptosis

Administration of Poly(I:C) produced a dose dependent (0, 5, 15, 30, and 50 μg) increase in the number of cells recovered per ml of BAL fluid obtained from control animals. This dose dependent effect was significantly exacerbated in animals exposed to cigarette smoke at the 15, 30, and 50 μg doses of Poly(I:C). Measures of differential cell recovery indicate that macrophage, lymph, and meutrophil cell numbers were all significantly increased in BAL recovered from animals exposed to CS.

Histological indications of inflammation are increased in animals exposed to either Poly(I:C) or CS alone as compared to normals, but the number of invading inflammatory cells and the degree of tissue remodeling is more prominent in animals administered Poly(I:C) and exposed to CS, resulting in the induction of emphysema as measured by changes in lung morphology and morphometry. The mean chord length for animals administered Poly(I:C) and exposed to CS was significantly increased to about 90 microns as compared to control animals with a mean chord length of about 55 microns.

Experiments which examined the effect of LPS and CS on BAL inflammation and lung tissue remodeling demonstrated that while LPS induced BAL inflammation in terms of the number of cells recovered in BAL fluids in a dose dependent manner (0, 1, and 10 μg doses of LPS), there was not a significant difference in animals exposed to CS and controls. In addition, no tissue remodeling was observed in these experiments.

Similar experiments were carried out using 0, 5, and 50 μg doses of GDQ. In control animals 5 μg doses of GDQ had no effect on BAL inflmation, while 50 μg doses of did. In animals exposed to CS, there was an overall increase in BAL inflammation that was not significantl between animals administered 0, 5, or 50 μg doses of GDQ. Further, GDQ had no effect on tissue remodeling in either control or CS animals.

Cell apoptosis, measured by TUNEL staining, is significantly increased in animals exposed to either CS (TUNEL score about 4) or Poly(I:C) (TUNEL score about 8), but the effect is dramatically increased when these two treatments are combined (TUNEL score about 17).

### Example 2

### Regulation of Type 1 Cytokines and Type I IFN by CS and Poly(I:C)

Four doses of Poly(I:C) administered sequentially over time increased the expression of IL-18, IFN-α/β, IL-12/23, and IFN-γ in a time-dependent manner in all control animals. In animals also exposed to CS, the increase in these cytokines was significantly enhanced.

IL-18 expression in control (non-CS) animals was significantly increased at the time of the second dose of Poly(I:C) from about 20 pg/ml to about 100 pg/ml in CS animals. By dose 3, this difference had narrowed, and by week 4 both CS and non-CS animals had IL-18 levels about 90-100 pg/ml after which time, IL-18 levels declined.

IL-12/23 levels were consistently elevated in CS vs non-CS animals with the greatest difference apparent after the second dose of Poly(I:C) when the IL12/23 in non-CS animals was about 3000 pg/ml but about twice that in CS animals.

IFN-α/β only exhibited an increase after the first dose of Poly(I:C) when control animals were about 50 U/ml and CS animals were about 350 U/ml. IFN-α/β levels then declines back to baseline and subsequent doses of Poly(I:C) had no effect.

IFN-γ levels became elevated in both non-CS and CS animals only after they were administered the second dose of Poly(I:C) and remained elevated. However, the CS animals IFN-γ levels were significantly higher than non-CS animals. The greatest difference was apparent after the second dose of Poly(I:C) when IFN-γ levels were about 10 pg/ml in control animals and about 90 pg/ml in CS animals. While IFN-γ levels peaked in control animals after the third dose of Poly(I:C), tapering off subsequently, CS animals continued to exhibit a rise in IFN-γ levels throughout the course of the experiment.

Animals wherein IL-18Rα and IFN-γ expression was ablated (IL-18Rα^{-/-} and IFN-γ^{-/-}, respectively) exhibited an insignificant decrease in the numbers of cells recovered from BAL fluid. However, both IL-18Rα^{-/-} and IFN-γ^{-/-} animals exhibited significantly less CS induced BAL inflammation.

Wild-type and IL-18Rα^{-/-} and IFN-γ^{-/-} animals administered Poly(I:C) all had mean chord lengths of about 60 microns. Wild-type administered Poly(I:C) and exposed to CS had chord and exposed to CS exhibited significantly reduced alveolar remodeling as measured by significantly reduced chord length as compared to wild type animals (about 70 microns for both).

Exposure to CS significantly increased IFN-γ expression in wild-type animals administered Poly(I:C) from about 35 pg/ml to about 110 pg/ml. In IL-18Rα^{-/-} animals administered Poly(I:C), this CS-induced increase in IFN-γ expression was completely abrogated (IFN-γ levels equal to about 35 pg/ml).

### Example 3

### Tole of Toll-Like Receptor 3 (TLR-3) in Inflammation and Remodeling Induced by CS and Poly(I:C)

Overall BAL inflammation is significantly increased by CS for both TLR^{+/+} and TLR-3^{-/-} animals. However, TLR-3^{-/-} animals administered Poly(I:C) exhibit a consistent decrease in BAL inflammation in both CS and noCS conditions as compared to TLR^{+/+} animals. There was an insignificant difference in mean chord length between TLR^{+/+} and TLR-3^{-/-} animals regardless of CS exposure.

### Example 4

### CS and Poly(I:C) Activate Double-Stranded RNA-Dependent Protein Kinase (PK-R), which Plays a Role in BAL Inflammation

Phospho-PKR is modestly up-regulated in wild-type animals in the presence of either CS or Poly(I:C). When wild-type animals are administered Poly(I:C) and exposed to CS, there is further increase in phosphor-PKR expression.

IL-18Rα^{+/+}, IFN-γ^{+/+}, IL-18Rα^{-/-}, and IFN-γ^{-/-} animals were administered Poly(I:C) and were either exposed to room air or CS.

IL-18Rα^{+/+} and IFN-γ^{+/+} animals administered Poly(I:C) expressed phosphor-PKR. This expression was enhanced when these animals were exposed to CS.

Phospho-PKR expression was significantly reduced in IL-18Rα^{-/-} animals when they were administered Poly(I:C). This expression was restored by exposure to CS. Similarly, phospho-PKR expression was significantly reduced in IFN-γ^{-/-} animals when they were administered Poly(I:C). This expression was restored by exposure to CS.

PKR^{+/+} and PKR^{-/-} animals exhibit similar levels of BAL inflammation in response to Poly(I:C). In the presence of CS, the increase in BAL inflammation seen in PKR^{+/+} animals is significantly reduced in PKR^{-/-} animals. Interestingly, PKR^{+/+} and PKR^{-/-} animals administered Poly(I:C) do not have significantly different degrees of alveolar remodeling (mean chord length for PKR^{+/+} is about 65 microns and 62 microns for PKR^{-/-} animals). Exposure to CS increased the mean chord length to about 85 microns for PKR^{+/+} but the mean chord length for PKR animals administered Poly(I:C) and exposed to CS is about 70 microns, a significant inhibition of alveolar remodeling. Thus PKR exacerbates alveolar remodeling induced by CS and Poly(I:C).

### Example 5

### Roles of IL-18Rα and IFN-γ and PK-R in CS and Poly(I:C) Induction of Apoptosis

IL-18Rα^{+/+}, IFN-γ^{+/+}, PKR^{+/+}, IL-18Rα^{-/-}, IFN-γ^{-/-}, and PKR^{-/-} animals were administered Poly(I:C) and were either exposed to room air or CS and a TUNEL score was determined. In IL-18Rα^{+/+}, IFN-γ^{+/+}, PKR^{+/+} animals, Poly(I;C) alone produced a TUNEL score of about 8% and about 12% in the presence of CS. Both these values were decreased to comparable levels in IL-18Rα^{-/-}, IFN-γ^{-/-}, and PKR^{-/-} animals, but the effect was only significant for animals who were exposed to both Poly(I:C) and CS. Specifically for −18Rα^{-/-}, IFN-γ^{-/-}, and PKR^{-/-} animals administered Poly(I:C), the TUNEL value was about 6.5%, but for −18Rα^{-/-}, IFN-γ^{-/-}, and PKR^{-/-} animals also exposed to CS, the TUNEL score was about 9%.

### Example 6

### Interaction of CS and Influenza A in the Induction of BAL Inflammation

Animals were infected with influenza A and either exposed to room air or CS for 3 to 15 days post-infection. Over this period, BAL inflammation increases for both non-CS and CS animals, peaking at 9 days post-infection before returning to baseline. Animals who were exposed to CS have significantly increased numbers of cells in BAL fluid at days 6, 9, and 12 as compared to non-CS animals. At day 9, the peak of Bal inflammation, non-CS animals had a mean of about 600×10³ cells/ml while CS animals had 1000×10³ cells/ml.

Measures of alveolar remodeling showed that CS alone did not produce a difference in mean chord length measured in animals not infected with influenza A. Similarly, animals infected with influenza A, but not exposed to CS did not differ in chord length from controls. But animals that were infected with influenza A exhibited a significant increase in chord length from about 57 microns (non CS) to about 70 microns (CS) when they were exposed to CS. Thus, the induction of alveolar remodeling is greatly exacerbated by viral infection in the presence of CS.

TUNEL measurements show that in the presence of influenza A viral infection, apoptosis increases from day 3 (about 3%) to day 9 post-infection (about 7%), but declines back to about 2% on day 15. In the presence of CS, TUNEL scores are all increased to about 4% (day 3), about 9.5% (day 9), and about 5.5% *day 15). In the presence of Influenza A infection from day 3 to day 15, PKR expression is fairly stable regardless of exposure to CS or not. elF2a expression in the absence of CS is reduced on day 9, but is comparable on days 3 and 15. Thus elF2a expression is inversely correlated with the time course of BAL inflammation, IL-18 and IFN-γ expression, and cell apoptosis during influenza A infection. Interestingly, this reduction in elF2a expression on day 9 post infection is reduced in the presence of CS. Similarly, elF2a expression levels aer elevated on day 15 post virus infection in the presence of CS.

### Example 7

### Cigarette Smoke (CS) Selectively Enhances Viral Pamp and Virus-Induced Pulmonary Innate Immune and Remodeling Responses

It has been shown that viral infections have more severe consequences in patients that have been exposed to cigarette smoke (CS). This is seen in chronic obstructive pulmonary disease (COPD) where viruses cause more severe disease exacerbations, heightened levels of inflammation and accelerated loss of lung function compared to other disease exacerbators. This attribute is also seen in the enhanced symptomatology and mortality in influenza-infected smokers.

The following experiments were designed to determine whether the aforementioned outcomes are the result of CS-induced alterations in innate immunity. Briefly, the effects of CS on pathogen-associated molecular pattern (PAMP)-induced pulmonary inflammation and remodeling were initially defined. These studies demonstrated that CS enhances viral PAMP (double stranded RNA; Poly(I:C))-induced parenchymal and airway inflammation and apoptosis, and that CS plus Poly(I:C) induce accelerated emphysema and airway fibrosis. This interaction was associated with the early induction of type I interferon (IFN) and IL-18, later induction of IL-12/23 p40, and IFN-γ as well as the activation of double-stranded RNA-dependent protein kinase (PKR) and eukaryotic initiation factor-2α (eIF2α). It was mediated by Toll-like receptor-3-dependent and independent, and mitochondrial anti-viral signaling (MAVS) protein-, IL-18Rα-, IFN-γ-and PKR-dependent mechanism(s). Furthermore, it was observed that CS interacted with influenza but did not interact withother innate immunity agonists in a similar fashion. These studies demonstrate that CS selectively augments viral PAMP- and virus-induced airway and alveolar inflammatory and remodeling responses in murine lung.

The materials and methods employed in these experiments are now described.

### Materials and Methods

Reagents

poly(I:C) was purchased from from Amersham Biosciences (Piscataway, N.J.) and lipopoly-saccharide (LPS), CpG, guardiquimod (GDQ) and imiquimod (IMQ) from Invivogen Inc. (San Diego, Calif.). Anti-IL-18 antibody (sc-7954) was purchased from SantaCruz for immunohistochemistry (IHC). For immunonlot analysis, antibodies against β-actin, caspase-3, PKR, PARP, eIF2α and phospho-eIF2α were purchased from Cell Signaling Technology (Danvers, Mass.). Phospho-PKR antibody was from Sigma-Aldrich (St. Louis, Mo.). The IFN-α/β standard for the type I IFN bioassay was purchased from Access Biomedical Diagnostic (San Diego, Calif.). For double-lebeling with TUNEL staining, anti-CC-10 and anti-CD31 were purchased from SantaCruz and anti-Pro SPC was from Chemicon.

Mice

C57BL/6J WT, IL-18Rα^{-/-} and IFN-γ^{-/-} mice were obtained from Jackson Labs (Bar Harbour, Ma.). PKR^{-/-} mice were provided by Cleveland Clinic Foundation. TLR-3^{-/-} mice were provided by Yale University.

Cigarette Smoking Exposure

Ten week old mice were exposed to RA or the smoke from non-filtered research cigarettes (2R4, University of Kentucky) using the smoking apparatus described by Hautamaki et al., 1997 Science 277: 2002-2004. During the first week they received a half cigarette, twice a day, to allow for acclimation. During the remainder of the exposure they received three cigarettes per day (one cigarette per session/three sessions per day).

In Vivo Administration of PAMPs

After two weeks of CS exposure the mice were anaesthetized and 50 μl aliquots of various PAMPs (poly(I:C), LPS, CpG, GDQ and IMQ) or their vehicle controls were administered twice per week (every Monday and Thursday) for two weeks (day 15, 18, 22 and day 25, respectively). CS exposure was continued during this interval. At the desired time point the mice were sacrificed and evaluated. For the experiments, mice were sacrificed one day after each administration.

In Vivo Administration of Influenza Virus

After two weeks of CS exposure the mice were anaesthetized and Influenza virus (A/PR8/34) (1/20 of LD₅₀) was administered using techniques previously described by Liu et al., 2005 Am J Respir Cell Mol Biol 33:463-469.

Bronchoalveolar Lavage (BAL)

Mice were sacrificed using intraperitoneal ketamine/xylazine injection and the trachea was cannulated and perfused with two 0.9 ml aliquots of cold saline. The cellular contents and BAL fluid were separated by centrifugation and the BAL fluid was stored in aliquots at −80° C.

Measurement of Lung Volume and Lung Morphometry

After BAL, the lungs were removed en block and inflated with PBS at the pressure of 25 cm. After complete inflation, the size of the lung were evaluated via volume displacement. The right main bronchus was ligated and the right lung was removed to be saved for molecular work. The left lung was inflated with 0.5% low temperature-melting agarose in STRECK fixative at a constant pressure of 25 cm. This allowed for homogenous expansion of lung parenchyma as described by Halbower et al. 1994, Lab Invest 71: 149-153. The lungs were then fixed, paraffin-embedded and H&E stained. Ten random fields were evaluated by microscopic projection onto NIH Image program (ver. 1.63) and alveolar size was estimated from the mean chord length of the air space as described elsewhere herein.

Quantification of BAL Fluid Cytokines

The levels of BAL IL-18 (MBL, Japan), IL-12/IL-23p40 and IFN-γ (R&D Systems) were determined using commercial ELISA kits as per the manufacturer's instructions.

IFN-α/β Bioassay

Samples were acid treated to a pH of 2 to inactivate any input virus as well as other cytokines. The samples were then neutralized with sodium bicarbonate, and twofold dilutions of each test sample were added to murine fibroblast monolayers in 96-well plates. After overnight incubation at 37° C., 1.25×10⁵ PFU of vesicular stomatitis virus (VSV) were added to each well. An IFN-α/β standard was used in parallel to generate a standard curve. Additional controls included untreated monolayers with and without VSV infection. After 2 days of incubation, wells were fixed with 2% formaldehyde and stained with crystal violet. Test sample IFN-α/β concentrations were determined by comparison of protection from VSV-induced cell killing with that seen with known amounts of IFN-α/β.

Immunohistochemistry (IHC) of IL-18

IHC of IL-18 was undertaken as described elsewhere herein.

TUNEL Analysis

End labeling of exposed 3′-OH ends of DNA fragments in paraffin embedded tissue was undertaken with the TUNEL in situ cell death detection kit AP (Roche Diagnostics, Indianapolis, Ind.) using the instructions provided by the manufacturer. Staining specificity was assessed by comparing the signal that was seen when terminal transferase was included and excluded from the reaction. After staining, a minimum of 10 fields of alveoli was randomly chosen and 300 nuclei of structural cells were counted per lung. The labeled cells were expressed as a percentage of total nuclei.

Immunoblot Analysis

Whole lung lysates were prepared and the total protein content of each was measured using the DC protein assay reagents (Bio-Rad Inc.). Equal amounts of sample proteins were fractionated on 4-15% SDS-PAGE gels under reducing conditions and transferred to polyvinylidene difluoride membranes. The membranes were then incubated in blocking buffer (5% w/v nonfat dry milk in TBS/0.05% Tween) for 1 hour at room temperature. The membranes were then incubated with primary antibodies overnight at 4° C., washed three times in TBS/0.05% Tween and incubated for 2 h at room temperature with appropriate secondary antibodies. Immunoreactive signal was detected using a chemiluminescent procedure (ECL Western blotting detection system; Amersham Life Science) according to the manufacturer's instructions.

Double Labeling of TUNEL and Structural Cell Markers

After TUNEL staining described as above, IHC was undertaken to colocalize CC-10, CD31, and Pro-SPC (+) cells with TUNEL(+) cells. Anti-CC-10, anti CD31 and anti-Pro-SPC were applied at 1:1,000, 1:1,000 and 1:500 respectively and incubated overnight at 4° C. CC-10 and CD31 were developed with a donkey anti-goat alexa 555 (Molecular Probes) and Pro-SPC was developed with a donkey anti-rabbit alexa 555 (Molecular probes). Mounting was with Vector Shield (Vector). Images were photographed on an Olympus BH-2 fluorescent microscope and analyzed using ImageJ software.

Statistical Analyses

Statistical evaluations were undertaken with SPSS software. As appropriate, groups were compared with Student's t test or with nonparametric Mann-Whitney U test. Values are expressed as mean±S.E.M. Statistical significance was defined at a level of p<0.05.

The results of the experiments are now described.

### Inflammatory Effects of Poly(I:C) in CS-Exposed Mice

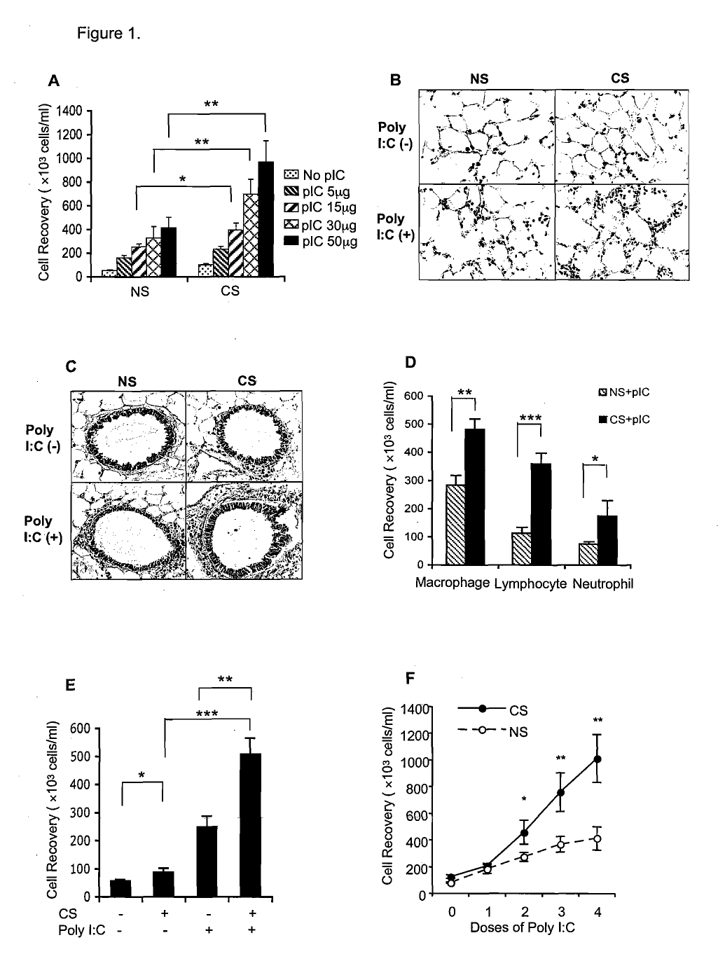

To evaluate the effects of CS on innate immune responses in the lung, experiments were designed to assess the responses induced by ligands that activate known innate immune pathways in C57BL/6J mice breathing RA and mice exposed to CS for varying intervals. Double-stranded RNA (dsRNA; Poly(I:C)) was initially chosen in an attempt to define responses that could be elicited by viral infections. CS exposure caused modest increases in BAL total cell and neutrophil recovery (FIG. 1A). It was also observed that CS exposure caused scattered foci of neutrophilic inflammation in lungs from some, but not all mice (FIGS. 1B and C). In contrast, Poly(I:C) caused significant increases in total cell, macrophage, lymphocyte and neutrophil recovery, as well as a neutrophil and macrophage-rich tissue inflammatory response (FIGS. 1A-1D). Importantly, CS and Poly(I:C) interacted in a synergistic manner to enhance this BAL, airway and alveolar inflammation (FIGS. 1A-1D). Similar responses were observed in Balb/c mice (FIG. 1E). This interaction was dose-dependent with striking effects observed when 50 μg of Poly(I:C) (the highest dose tested) was given to CS-exposed mice for 2 weeks (4 doses) (FIGS. 1A and 1F). Significant interactions were also seen with doses of Poly(I:C) as low as 15 μg and after 2 aspirations of high dose (50 μg) Poly(I:C) (FIGS. 1A and 1F). Importantly, this interaction was also time-dependent with exposures as short as 2 weeks and as long as 3 months causing comparable increases in Poly (I:C)-induced inflammation and lesser effects observed with shorter intervals of CS exposure. In all cases, this effect was at least partially Poly(I:C)-specific because similar synergy was not observed when LPS (0.01-10 μg), CpG (0.5-5 μg), gardiquimod (GDQ, 5-50 μg) or imiquimod (IMQ, 10-100 μg) were administered in a similar fashion. It also required CS and Poly (I:C) because LPS did not interact in a similar manner with Poly (I:C) and the repeated administration of Poly (I:C) alone for up to 3 months did not induce comparable tissue effects. These results demonstrate that CS selectively enhances the alveolar and airway inflammatory effects of Poly(I:C) in the murine lung.

### Remodeling Responses Induced by Poly(I:C) in CS-Exposed Mice

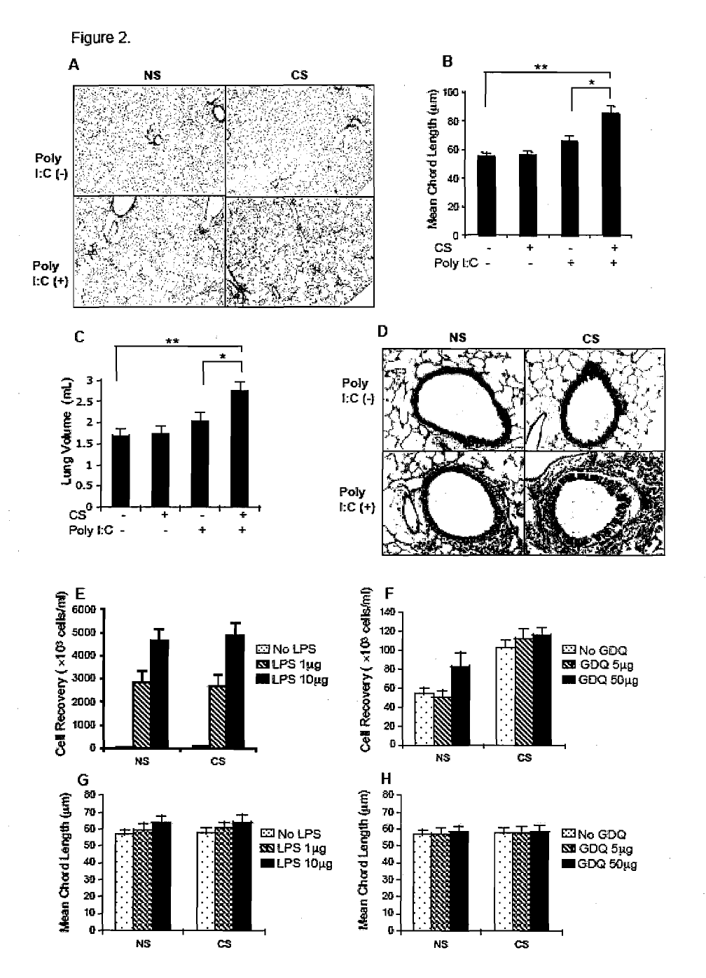

To gain insight into the functional consequences of the interaction observed above, lung tissues in C57BL/6J mice breathing RA and mice that had been exposed to CS for 2 weeks prior to Poly(I:C) administration were evaluated. At this time point, CS alone did not cause a significant increase in alveolar size and 4 doses of Poly(I:C) in RA-exposed mice caused an increase that did not achieve statistical significance (FIGS. 2A-2B). In contrast, impressive alveolar remodeling with emphysematous alterations on light microscopic examination and significant increases in morphometrically determined alveolar chord length were observed in CS-exposed mice that received 4 doses of Poly(I:C) (FIGS. 2A and 2B). The lungs from mice exposed to CS plus Poly (I:C) also manifest increased lung volumes after pressure fixation (FIG. 2C). Similar responses were also observed in Balb/c mice. In addition, these alveolar alterations were associated with a fibrotic airway remodeling response that was detectable on trichrome histologic evaluation (FIG. 2D). In all cases these interactions were dose- and time dependent and could be appreciated with doses of Poly(I:C) as low as 15 μg and after as few as 2 doses of high concentration Poly(I:C). They were also at least partially Poly(I:C)-specific because LPS, CpG, GDQ and IMQ did not have similar effects (FIGS. 2E-2H). In addition, they also required CS plus Poly (I:C) because LPS did not interact in a similar fashion with Poly (I:C) and repeat dosing with Poly (I:C) alone for up to 3 months did not induce similar remodeling responses. Thus, in addition to its ability to enhance inflammation, CS selectively enhances the ability of Poly(I:C) to induce pulmonary emphysema and airway remodeling.

### Regulation of IL-18, IL-12 and Type I and Type II Interferons (IFNs)

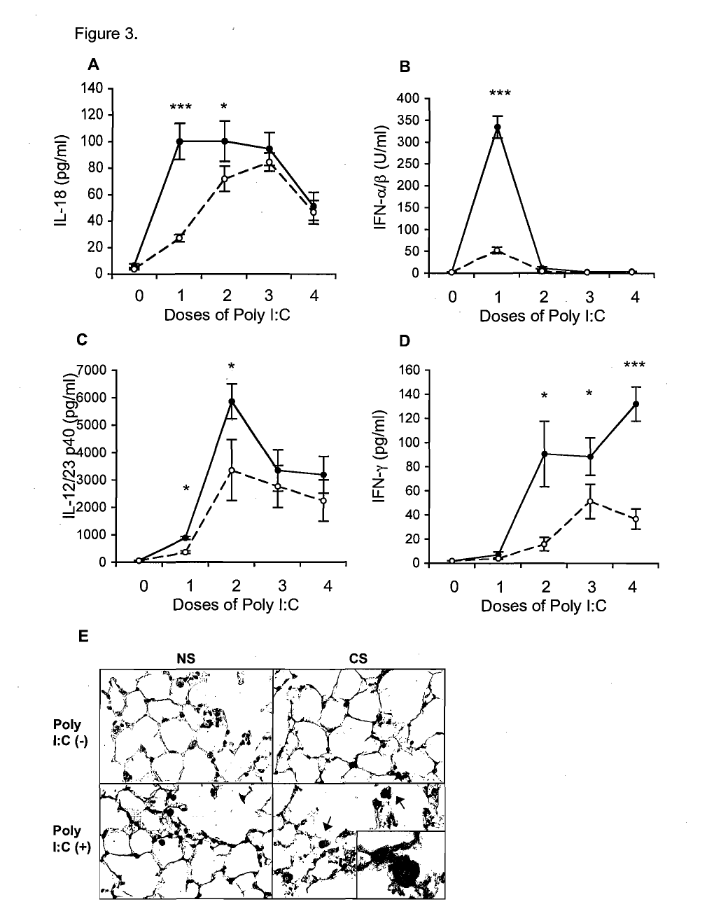

In keeping with their roles in viral responses and COPD, the next set of experiments were designed to evaluate the production of IL-18, IL-12/23 p40 and Type I and Type II IFNs in this modeling system. In these experiments, the vehicle controls did not stimulate the production of any of these cytokines. In contrast, Poly(I:C) was a potent stimulator of IL-18, IL-12/23 p40 and Type I and Type Il IFNs (FIGS. 3A-3D). Importantly, these inductive responses were significantly greater in mice exposed to CS compared to mice in RA (FIGS. 3A-3D). In all cases, these inductive events had distinct kinetic patterns with IL-18 and Type I IFNs being induced early (after a single dose of Poly(I:C)), IL-12/23 p40 being observed with an intermediate kinetic (after 2-3 doses of Poly(I:C)), and IFN-γ being induced later (after 3-4 doses of Poly(I:C)) (FIGS. 3A-3D). Similar inductive responses were not seen when CS-exposed animals were treated with LPS, CpG, GDQ or IMQ. Thus, the ability of CS to selectively enhance Poly(I:C)-induced inflammation and alveolar remodeling is associated with the enhanced production of IL-18, IL-12/23 p40, IFN-γ and Type I IFNs.

To further define these cytokine responses, immunohistochemistry was used to localize the IL-18 in the modeling system. IL-18 was detected in airway epithelial cells and alveolar type 2 cells in lungs from WT mice (FIG. 3E). Low levels of IL-18 were also appreciated in alveolar macrophages from these animals (FIG. 3E). In mice exposed to CS plus Poly (I:C), increased levels of IL-18 were appreciated. This was more prominent in the alveolar macrophages with lesser increases being noted in the alveolar and airway epithelial populations (FIG. 3E). This demonstrates that alveolar macrophages are the major site of IL-18 augmentation in CS plus Poly (I:C)-treated mice.

### Roles of IL-18 Receptor (R) α and IFN-γ

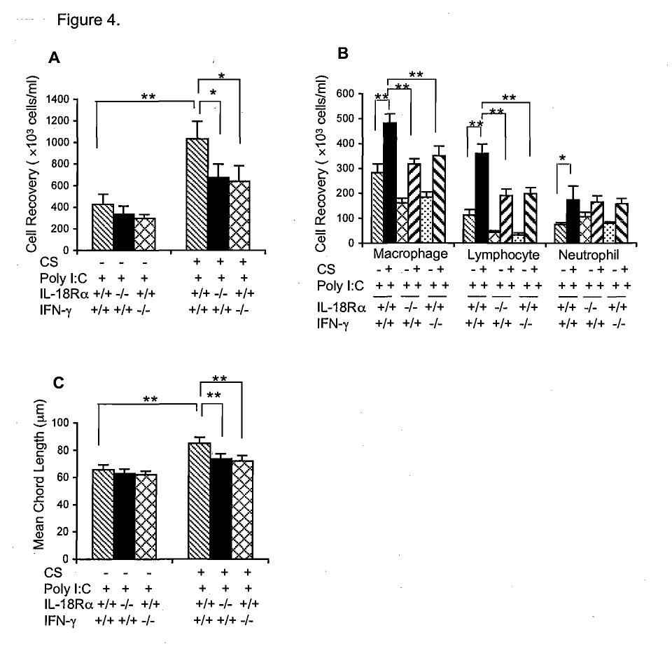

The roles of IL-18 and IFN-γ were subsequently evaluated by comparing the inflammatory, emphysematous and cytokine responses induced by CS plus Poly(I:C) in mice with wild type (WT) and null IL-18Rα or IFN-γ loci. In these experiments, CS plus Poly(I:C) induction of BAL inflammation, macrophage and lymphocyte accumulation, tissue inflammation and alveolar remodeling were significantly diminished in mice with null mutations of IL-18Rα or IFN-γ (FIGS. 4A-4C). Thus, IL-18Rα and IFN-γ play critical roles in the pathogenesis of the inflammation and remodeling that is induced by CS plus Poly(I:C).

### Cytokine-Cytokine Interactions

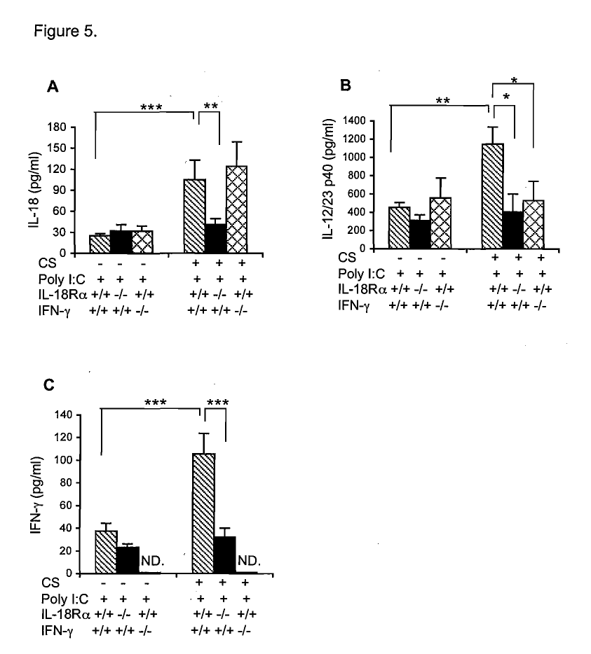

The next set of experiments were designed to define the relationships between the cytokines induced in this modeling system. It was observed that in the absence of IL-18Rα, the ability of CS plus Poly(I:C) to stimulate the production of IL-18, IL-12/23 p40 and IFN-γ were significantly diminished (FIGS. 5A-5C). In contrast, in the absence of IFN-γ, the induction of IL-18 was not altered while the induction of IL-12/23 p40 was significantly decreased (FIGS. 5A and 5B). These studies highlight a cytokine-receptor cascade in which IL-18 receptor signaling is a proximal event that plays a critical role in the induction of IL-12/23 p40 and IFN-γ, and that IL-18 is auto-induced via an IL-18Rα-dependent mechanism. The results also demonstrate that IFN-γ contributes to the induction of IL-12/23 p40 but not IL-18.

### Roles of TLR-3

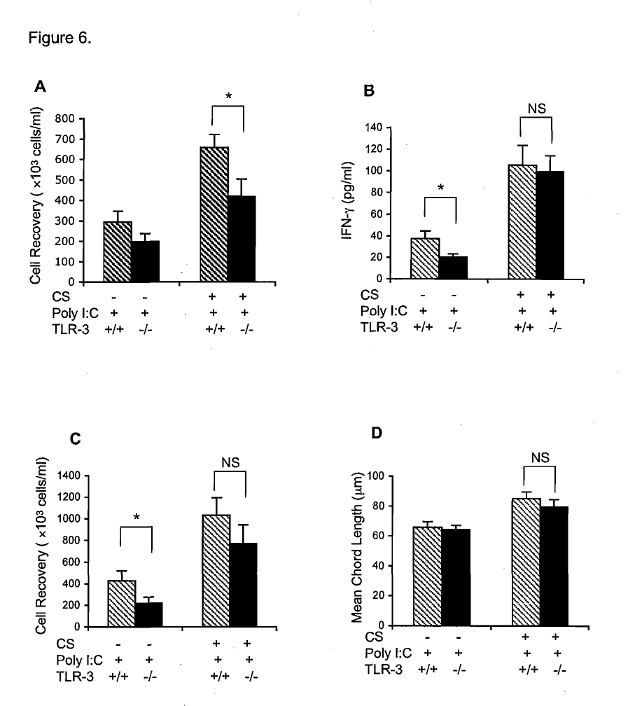

The next set of experiments were designed to define the innate immunity pathway(s) that is involved in the interaction of CS and Poly(I:C). Experiments were designed to evaluate the consequences of an acute challenge with Poly(I:C) (idose) or repeated challenges with Poly(I:C) (4 doses) in mice exposed to RA and CS with wild type and null TLR-3 loci. In mice given 1 dose of Poly(I:C) and evaluated 4 days later, a modest decrease in BAL and tissue inflammation was noted in comparisons of TLR-3 deficient and WT animals (FIG. 6A). In contrast, TLR-3 null and WT animals exposed to CS and 4 doses of Poly(I:C) had a similar ability to stimulate IFN-γ, induce BAL and tissue inflammation, and induce alveolar remodeling (see FIGS. 6B-6D). Thus, TLR-3 plays a role in the pathogenesis of the acute responses induced by Poly(I:C) in CS-exposed mice while repeated doses of Poly(I:C) induce inflammation and alveolar remodeling via a pathway(s) that is largely TLR-3-independent.

### Role of MAVS in Responses Induced by CS Plus Poly(I:C)

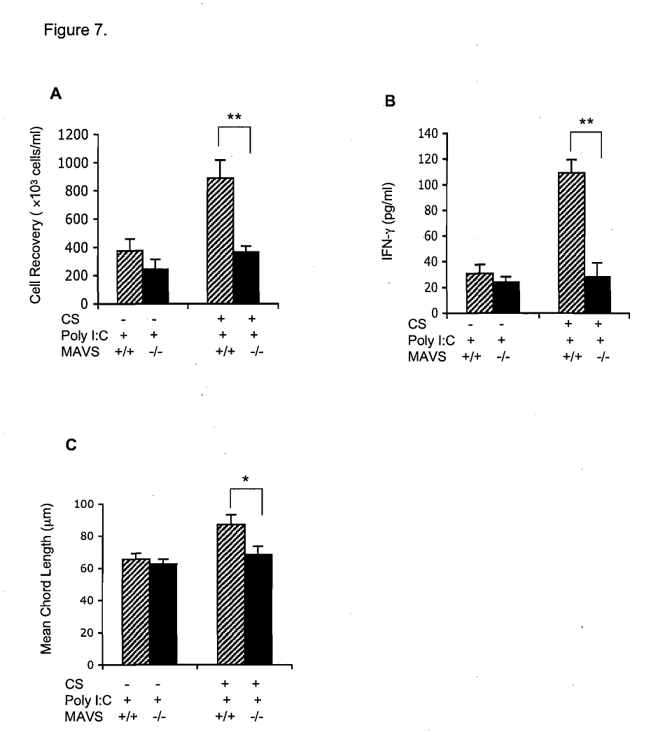

To further define the innate pathways that mediate the interactions of CS plus Poly(I:C), experiments were designed to compare the inflammation, cytokine responses, and alveolar remodeling in WT mice and mice with null mutations of the mitochondrial anti-viral signaling protein (MAVS). MAVS was chosen because it links RNA helicases like retinoic acid inducible gene-1 (RIG-1) and melanoma differentiation antigen 5 (Mda5) to anti-viral effector responses. These studies demonstrate that MAVS plays a critical role in these responses because the inflammatory, cytokine-inductive, and remodeling responses induced by Poly(I:C) in CS-exposed mice were abrogated in MAVS deficient animals (FIGS. 7A-7C). This demonstrates that a MAVS-related pathway plays a major role in the interaction of CS and Poly(I:C).

### Regulation and Roles of PKR

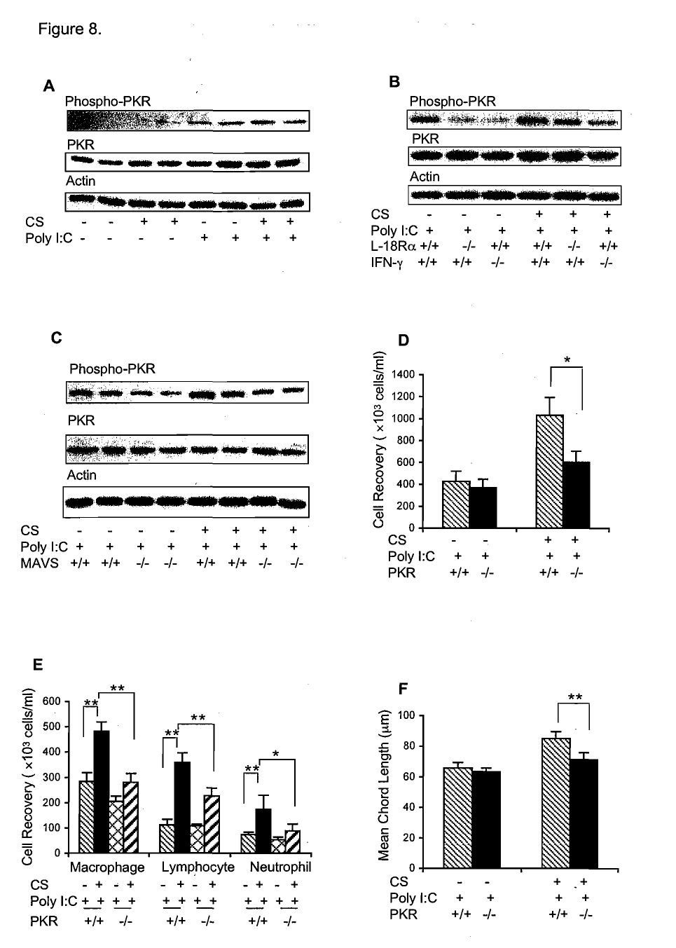

The next set of experiments were designed to evaluate the activation of PKR in the system. As shown in FIG. 8A, phosphorylated PKR was not appreciated or was present in only modest quantities in lungs from mice in RA, mice exposed to CS only, and mice treated with just Poly(I:C). In contrast, the activation of PKR was greatly enhanced in mice exposed to CS prior to Poly(I:C) administration (FIG. 8A). These inductive events were dose- and time-dependent, being observed after as little as 2 inhalations of high dose Poly(I:C) and with doses of Poly(I:C) as low as 15 μg/ml. They were also mediated by pathways that are at least partially IL-18Rα- and IFN-γ-dependent, because PKR phosphorylation was significantly diminished in IL-18Rα null and IFN-γ null animals (FIG. 8B). These activation events were also downstream of MAVS because the ability of Poly(I:C) to activate PKR in CS exposed mice was significantly decreased in MAVS null mice (FIG. 8C).

The functional consequences of PKR activation were next evaluated by comparing the effects of CS plus Poly(I:C) in mice with WT and null PKR loci. These studies demonstrated that the inflammatory and alveolar remodeling effects of Poly(I:C) in CS-exposed mice were significantly diminished in animals with null mutations of PKR (FIGS. 8D-8F). In contrast, null mutations of PKR did not alter the production of IL-18 or IFN-γ in mice treated with CS plus Poly(I:C). When viewed in combination, these studies demonstrate that PKR is activated via a MAVS-, IL-18Rα- and IFN-γ-dependent mechanism(s) in CS-exposed mice treated with Poly(I:C) and that this activation plays an essential role in the pathogenesis of the enhanced inflammatory and emphysematous responses in these animals. They also demonstrate that PKR does not play a role in the enhanced induction of IL-18 or IFN-γ, suggesting that PKR activation is distal to IFN-γ in this effector cascade.

### Apoptosis in Poly(I:C)-Treated CS-Exposed Animals

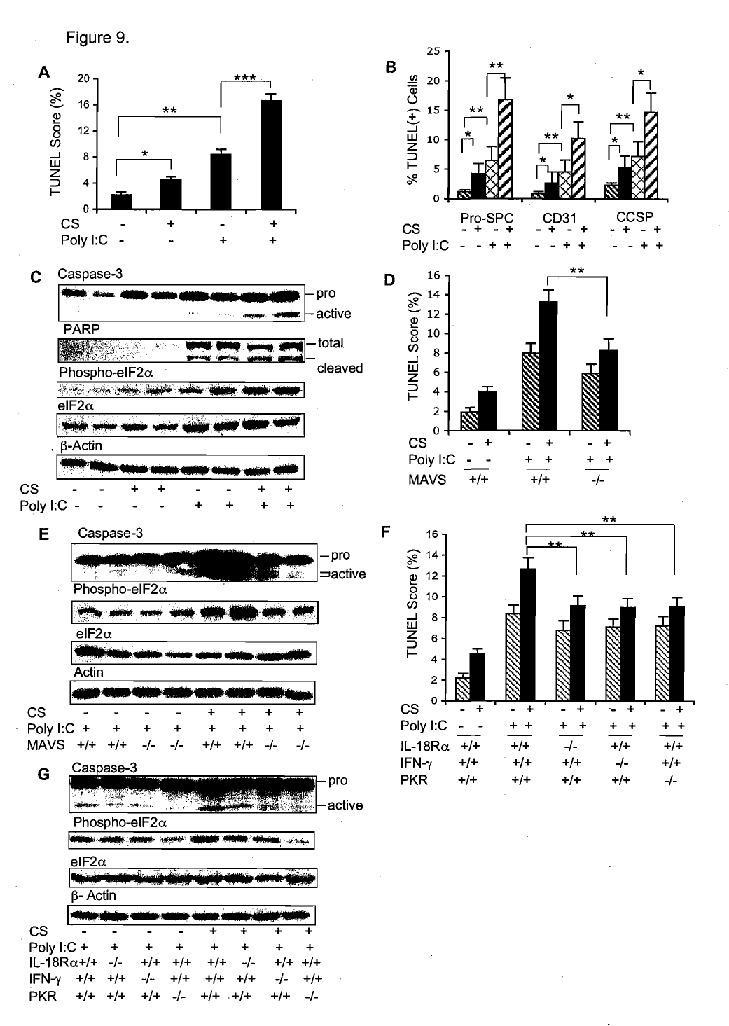

In keeping with the importance of structural cell apoptosis in CS-induced emphysema, the next set of experiments were designed to evaluate the DNA injury and cell death responses in the system. These studies demonstrated that CS alone and Poly(I:C) administration to mice breathing RA each caused small, but significant, increases in the number of cells that stain positive for TUNEL (FIG. 9A). In contrast, a significant increase in the number of TUNEL positive cells was seen in the parenchyma and airways of CS-exposed mice that were treated with Poly(I:C) (FIG. 9A). Double label immunohistochemistry demonstrated significant increases in the percentage of pro-SP-C (+) alveolar epithelial cells, CD31 (+) endothelial cells and CCSP (+) airway epithelial cells in lungs from mice incubated with CS plus Poly (I:C). (FIG. 9B). In contrast, less than 20% of the TUNEL cells were CD3(+) or F4/80 (+). Caspase-3 activation, cleavage of the caspase target poly (ADP-ribose) polymerase (PARP) and phosphorylation of eukaryotic initiation factor-2((eIF2α), a major PKR target that plays an important role in a variety of apoptotic responses, were not observed or were weakly detectible in lungs from mice breathing RA, CS-exposed mice and mice that received only Poly(I:C) (FIG. 9C). In accord with the TUNEL results, each of these responses was detected in significantly enhanced quantities in mice exposed to CS plus Poly(I:C) (FIG. 9C). This DNA injury and cell death response was mediated via a MAVS-, IL-18Rα-, IFN-γ- and PKR-dependent mechanism(s) since the levels of TUNEL staining, eIF2α phosphorylation and caspase-3 activation were significantly diminished in Poly(I:C) plus CS-treated mice with null mutations of MAVS, IL-18Rα, IFN-γ or PKR, respectively (FIGS. 9D-9G). These studies demonstrate that CS enhanced the ability of Poly(I:C) to induce epithelial and endothelial cell DNA injury, cell death and caspase and eIF2α activation. The results also highlight important roles that MAVS, IL-18Rα, IFN-γ and PKR play in these apoptotic responses.

### Effects of Influenza in CS-Exposed Mice

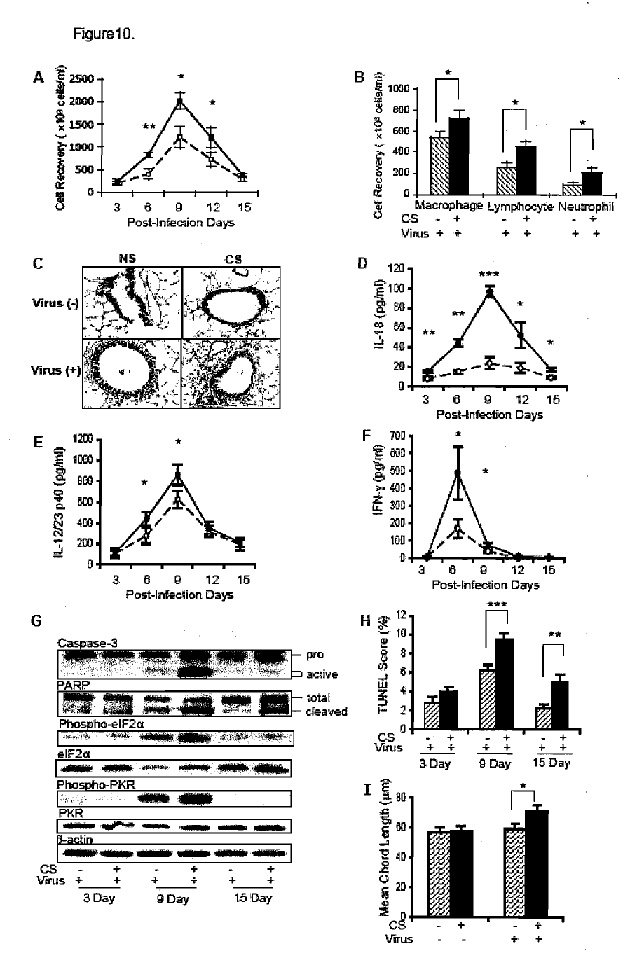

The studies discussed above used Poly(I:C) as a surrogate for double stranded RNA viruses and single stranded RNA viruses that go through a double stranded stage during viral replication. Therefore, the next set of experiments were designed to evaluate the validity of this concept. Experiments were designed to compare the effects of influenza virus in mice exposed to RA or CS for 2 weeks prior to infection. In mice breathing RA, influenza infection caused alveolar, airway and BAL inflammatory responses characterized by increases in total cell, neutrophil, lymphocyte and macrophage accumulation that peaked 9 days after inoculation (FIGS. 10A-10C). This response was associated with modest increases in the production of IL-18 (FIG. 10D), IL-12/23 p40 (FIG. 10E) and IFN-γ (FIG. 10F), modest levels of caspase 3 activation, PARP cleavage and PKR and eIF2α activation (FIG. 10G) and low levels of TUNEL staining (FIG. 10H). Emphysematous alveolar remodeling was not observed (FIG. 10I). In accord with the results presented herein with Poly(I:C), each of these responses were exaggerated in CS-exposed mice infected with influenza (FIGS. 10A-10H). Influenza infection also induced emphysematous alveolar remodeling and increased the levels of apoptosis in SP-C (+) and CD31(+) cells in CS-exposed animals (FIG. 10I). These studies demonstrate that CS enhanced the inflammatory, remodeling and apoptotic effects of influenza in the lung and highlight the induction of IL-18, IL-12/23 p40, and IFN-γ and activation of PKR and eIf2α in this setting.

### Pathways in CS-Influenza Interaction

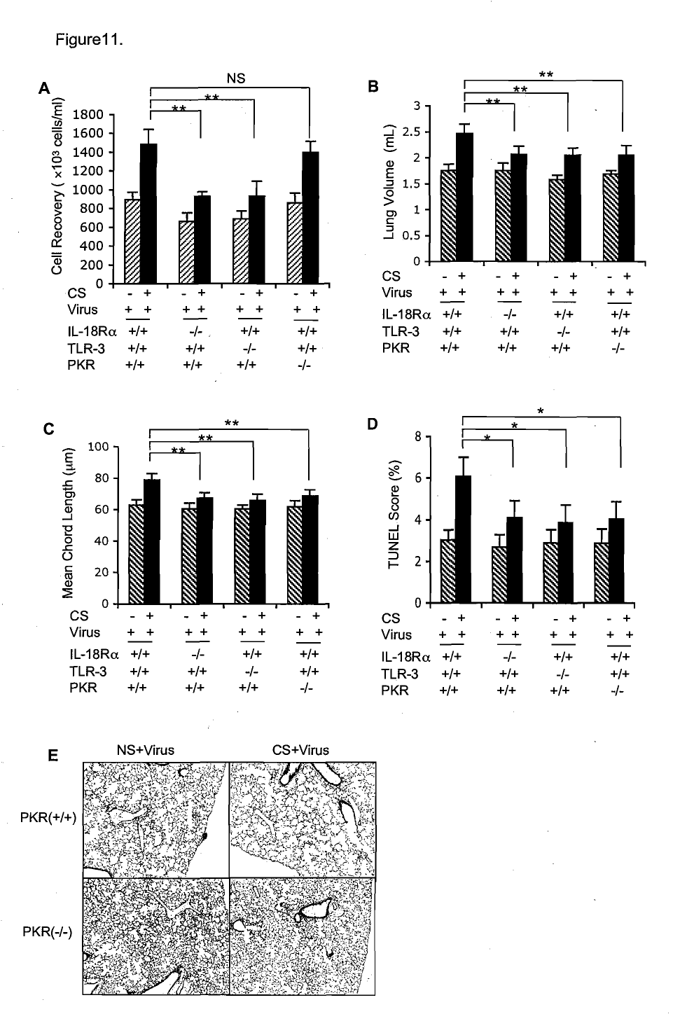

To define the mechanisms that underlie these responses, the next set of experiments were designed to compare the effects of influenza in CS-exposed mice with null mutations of IL-18Rα, TLR-3 and PKR. In accord with the results presented herein with Poly(I:C), it was observed that IL-18R signaling played an important role in the inflammation, alveolar remodeling and apoptosis induced by influenza in CS-exposed mice (FIGS. 11A-11D). TLR-3 also played a role in the inflammation and remodeling in CS-exposed, influenza-infected mice (FIGS. 11A-11D). Interestingly, the contributions of PKR were more complex with CS plus influenza-induced inflammation being mediated by PKR-independent and alveolar remodeling and apoptosis being mediated by PKR-dependent pathways (FIGS. 11A-11E). Importantly, these augmented virus-induced responses could not be attributed to differences in viral clearance because similar viral loads were seen in lungs from mice breathing RA and CS at all time points. These studies demonstrate that IL-18Rα, TLR-3 and PKR play important roles in the inflammatory and remodeling responses induced by CS plus influenza.

Mechanisms that can Contribute to the Pathgenesis of Virus-Induced COPD Exacerbations

The experiments discussed herein were designed to determine whether CS alters innate immune responses in the lung by comparing the inflammatory and remodeling responses induced by innate immunity agonist PAMPs in mice breathing RA and mice exposed to CS. The results disclosed herein demonstrate that CS has a remarkable and selective effect on these responses. For example, the results demonstrate in two different murine strains that CS selectively enhanced the airway and parenchymal inflammatory, remodeling, and apoptotic responses induced by the viral PAMP dsRNA. The results also demonstrate that this interaction is associated with the early induction of type I IFNs and IL-18, later induction of IL-12/23 p40 and IFN-γ, and the activation of PKR and eIF2α. The results also highlight the TLR 3-dependent and-independent, MAVS-dependent, and IL-18Rα-, IFN-γ- and PKR dependent mechanisms that contributes to these events. The viral relevance of these findings was also defined by demonstrating that CS interacts in a similar fashion with influenza to increase inflammation, apoptosis and emphysema, IL-18 and IFN-γ production and activate PKR. Furthermore, the results presented herein defined the important roles that TLR3, IL-18Rα and PKR play in these virus-CS-induced responses. These results are the first to define the CS-induced alterations in virus-related innate immune responses in the lung, the first to demonstrate that exaggerated inflammatory and remodeling responses are observed when CS and viral PAMPs or live viruses are combined, and the first to define the pathways that mediate these responses. These studies provide insight into the mechanisms that can contribute to virus-induced COPD exacerbations and the impressive symptomatology and loss of lung function that are noted in this setting. They also provide insight into the mechanisms that can contribute to the exaggerated severity of viral infections in otherwise healthy smokers and highlight targets against which therapies can be directed to control virus-induced responses in smokers with and without COPD.

In studies that have focused largely on bacterial stimuli, CS and its components like nicotine have been shown to have immunosuppressive properties including the ability to inhibit macrophage function (reviewed in Sopori, M., 2002 Nat Rev Immunol 2: 372-377). Surprisingly, these lesions have been extrapolated to viruses despite preliminary studies that suggest that viral responses may be different (Robbins et al., 2006 Am J Respir Crit Care Med 174: 1342-1351). The results presented herein demonstrate that the concept that CS is strictly immunosuppressive is not correct. Specifically, the results presented herein demonstrate that, in the case of viral PAMPs and live virus, CS enhances immune responses and the tissue consequences of these responses. They also highlight the impressive specificity of this interaction by demonstrating that a variety of other innate immunity agonists do not interact with CS in a similar manner, can not replace CS in this experimental system, and do not induce tissue remodeling if repeatedly administered. These studies provide an intriguing explanation for the increased incidence of symptoms in virus-infected smokers versus non-smokers, the enhanced severity of influenza in smokers versus non-smokers, and the finding that virus-induced COPD exacerbations are more severe, last longer, and are associated with greater inflammatory responses and loss of lung function than exacerbations due to other causes. Because these responses were observed in mice that were transiently exposed to CS, it is believed that these findings also have important implications with respect to the potential health consequences of second hand smoke and the mechanisms that may mediate the effects of smoke in this setting.

Historically, bacteria have been considered to be the main infectious cause of COPD exacerbations. However, modern PCR-based diagnostic approaches, have demonstrated that viruses are major contributors in 39% of outpatient exacerbations and a higher percentage of patients with severe exacerbations. The demonstration that CS selectively augments viral PAMP-induced and live virus-induced inflammation and remodeling has a number of implications with respect to the pathogenesis of and treatment of COPD. First, recent studies have demonstrated that RSV can be detected in the lower airway of patients with stable COPD and that these patients have higher levels of inflammation and an accelerated loss of lung function compared to RSV negative individuals (Seemungal et al., 2001 Am J Respir Crit Care Med 164: 1618-1623; Wilkinson et al., 2006 Am J Respir Crit Care Med 173: 871-876). Without wishing to be bound by any particular theory, since CS synergizes with RSV in a manner that is analogous to influenza, it is believed that this CSRSV interaction is responsible for the exaggerated responses in these patients. The results presented herein provide a strong rationale for thorough viral diagnostic evaluations in patients with COPD exacerbations and suggest that interventions that alter the activation of the MAVS, TLR-3, IL-18, IL-18Rα, IL-12/23p40, IFN-γ, PKR, eIF2α pathway(s) that mediates these responses may have therapeutic utility.

Host antiviral responses are initiated via the detection of viral PAMPs by host pattern recognition receptors (PRRs) (Kumar et al., 2006 J Exp Med 203: 1795-1803; Sun et al., 2006 Immunity 24: 633-642. Upon recognition, PRR signaling results in the expression of type I IFNs which suppress viral replication and facilitate adaptive immune responses. Double stranded RNA, which is produced during the replication of many viruses, is recognized by several innate pathways including TLR-3 and the RNA helicases RIG-1 and Mda5. In addition, many single-stranded RNA viruses, including influenza, have been shown to activate the RIG-I pathway via the generation of 5′-triphosphorylated single stranded RNA. TLR-3 resides in endosomal membranes where it recognizes dsRNA and Poly(I:C). RIG-1 and Mda5 detect dsRNA and Poly(I:C) in the cytoplasm where they are linked to downstream signaling molecules via MAVS.

Initially, it was hypothesized that the heightened inflammatory and remodeling responses induced by Poly(I:C) in mice exposed to CS would be mediated, in great extent, by TLR-3. However, the results presented herein demonstrate that this is not entirely correct because the acute and chronic inflammatory and remodeling responses induced by CS plus poly(I:C) were mediated by partially TLR3-dependent and TLR3-independent pathways respectively. In light of the limited roles of TLR-3 in these responses the contributions of the spiral helicase pathway were evaluated. These studies demonstrated that the synergistic interactions of Poly(I:C) and CS were significantly ameliorated in the absence of MAVS. They also demonstrated that PKR and eIF2α were activated by CS plus Poly (I:C) via a MAVS-dependent mechanism(s). These are the first studies to highlight the ability of CS to regulate the activation of this spiral helicase pathway. They also are the first to demonstrate that this helicase pathway can regulate the activation of PKR and eIF2α.

PKR is a serine threonine kinase (reviewed in Garcia et al., 2006, Microbiol Mol Biol Rev 70: 1032-1060) that was initially discovered as a IFN-induced and activated gene that mediated anti-viral effects largely via its ability to activate eIF2α and inhibit protein translation. It is now known that PKR is activated by viruses, dsRNA, cytokines, growth factors, oxidative stress and ceramide. After activation, PKR plays an important role in cell signaling and regulates gene transcription, at least in part, via its ability to interact with known signaling pathways including nuclear factor-κB (NF-κB), MAP kinases, p53, STATs and interferon regulatory factor 1 (IRF-1) (Garcia et al., 2006, Microbiol Mol Biol Rev 70: 1032-1060). As a result, it is now appreciated that PKR also plays an important role in the regulation of cell proliferation and differentiation, tumor suppression and cellular apoptosis.

The results presented herein demonstrate that CS interacts with viral PAMPs and live virus to activate PKR and eIF2α and that this induction is mediated via a MAVS-, IL-18Rα- and IFN-γ-dependent mechanism. The results presented herein also demonstrate that PKR plays an important role in the pathogenesis of the inflammatory, apoptotic and emphysematous responses induced by viral PAMPs and live virus in CS-exposed mice. These are the first studies to demonstrate that CS regulates the PKR pathway and the first studies to implicate PKR and eIF2α activation in the pathogenesis of the inflammation, apoptosis or emphysema in COPD. When viewed in combination, the results presented herein suggest that PKR and eIF2α are activated by a MAVS-IL-18-IFN-γ cascade in mice exposed to CS and viral PAMPs or live virus and that PKR and eIF2α contribute to the pathogenesis of pulmonary emphysema, at least in part, via their ability to induce pulmonary structural cell apoptosis.

In summary, the results presented herein demonstrate that CS selectively augments viral PAMP and live virus induced airway and alveolar inflammation and remodeling in the murine lung and highlight TLR-3-dependent and independent and MAVS/PKR/IL-18Rα/IFN-γ-dependent pathway(s) that contributes to the pathogenesis of these responses. The results presented herein provide insights into mechanisms that can contribute to the pathogenesis of virus-induced COPD exacerbations and lung function deterioration, the enhanced severity of viral infections in smokers, and the toxic effects of second hand smoke. They also identify new targets against which therapies can be developed to modulate the severity of virus-induced responses in these clinical settings.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of treating a disease associated with dysregulation of IL-18 expression in lung, said method comprising administering a therapeutically effective amount of at least one IL-18 inhibitor to a mammal having said disease wherein said IL-18 inhibitor attenuates, prevents, or halts the dysregulation of said IL-18 expression, thereby reducing said IL-18 expression in the lungs of said mammal.

2. The method of claim 1 wherein said IL-18 inhibitor comprisesan inhibitor selected from the group consisting of an antibody, siRNA, a ribozyme, an antisense, an aptamer, a peptidomimetic, a small molecule, and any combination thereof.

3. The method of claim 2, wherein said antibody comprises an antibody selected from the group consisting of a polyclonal antibody, a monoclonal antibody, a humanized antibody, a synthetic antibody, a heavy chain antibody, a human antibody, and a biologically active fragment of an antibody.

4. The method of claim 3, wherein said mammal is a human.

5. A method of treating a disease associated with dysregulation of IFNγ expression in lung, said method comprising administering a therapeutically effective amount of at least one IFNγ inhibitor to a mammal having said disease wherein said IFNγ inhibitor attenuates, prevents, or halts the dysregulation of said IFNγ expression, thereby reducing said IFNγ expression in the lungs of said mammal.

6. The method of claim 5 wherein said IFNγ inhibitor comprises an inhibitor selected from the group consisting of an antibody, siRNA, a ribozyme, an antisense, an aptamer, a peptidomimetic, a small molecule, and any combination thereof.

7. The method of claim 6, wherein said antibody comprises an antibody selected from the group consisting of a polyclonal antibody, a monoclonal antibody, a humanized antibody, a synthetic antibody, a heavy chain antibody, a human antibody, and a biologically active fragment of an antibody.

8. The method of claim 7, wherein said mammal is a human.

9. A method of treating a disease associated with dysregulation of double stranded RNA-dependent protein kinase (PKR) expression in lung, said method comprising administering a therapeutically effective amount of at least one PKR inhibitor to a mammal having said disease wherein said PKR inhibitor attenuates, prevents, or halts the dysregulation of said PKR expression, thereby reducing said PKR expression in the lungs of said mammal.

10. The method of claim 9 wherein said PKR inhibitor comprises an inhibitor selected from the group consisting of an antibody, siRNA, a ribozyme, an antisense, an aptamer, a peptidomimetic, a small molecule, and any combination thereof.

11. The method of claim 10, wherein said antibody comprises an antibody selected from the group consisting of a polyclonal antibody, a monoclonal antibody, a humanized antibody, a synthetic antibody, a heavy chain antibody, a human antibody, and a biologically active fragment of an antibody.

12. The method of claim 11, wherein said mammal is a human.

13. A method of inhibiting inflammation in the lung of a mammal at risk of developing inflammation, wherein said inflammation is the result of exposure to viral infection and cigarette smoke, said method comprising administering a therapeutically effective amount of an inhibitor to said mammal having said inflammation, wherein said inhibitor prevents said inflammation, and further wherein said inhibitor is selected from the group consisting of an IL-18 inhibitor, an IL-18Rα inhibitor, and IFNγ inhibitor, a PKR inhibitor, and any combination thereof.

14. The method of claim 13, wherein said inhibitor comprises an inhibitor selected from the group consisting of an antibody, siRNA, a ribozyme, an antisense, an aptamer, a peptidomimetic, a small molecule, and any combination thereof.

15. The method of claim 14, wherein said antibody comprises an antibody selected from the group consisting of a polyclonal antibody, a monoclonal antibody, a humanized antibody, a synthetic antibody, a heavy chain antibody, a human antibody, and a biologically active fragment of an antibody.

16. The method of claim 13, wherein said mammal is a human.

17. A method of inhibiting alveolar remodeling in the lung of a mammal at risk of developing alveolar remodeling wherein said alveolar remodeling is the result of exposure to viral infection and cigarette smoke, said method comprising administering a therapeutically effective amount of an inhibitor to said mammal having said alveolar remodeling, wherein said inhibitor prevents said alveolar remodeling, and further wherein said inhibitor is selected from the group consisting of an IL-18 inhibitor, an IL-18Rα inhibitor, and IFNγ inhibitor, a PKR inhibitor, and any combination thereof.

18. The method of claim 17, wherein said inhibitor comprises an inhibitor selected from the group consisting of antibody, siRNA, a ribozyme, an antisense, an aptamer, a peptidomimetic, a small molecule, and any combination thereof.

19. The method of claim 18, wherein said antibody comprises an antibody selected from the group consisting of a polyclonal antibody, a monoclonal antibody, a humanized antibody, a synthetic antibody, a heavy chain antibody, a human antibody, and a biologically active fragment of an antibody.

20. The method of claim 17, wherein said mammal is a human.

21. A method of inhibiting cellular apoptosis in the lung of a mammal at risk of developing cellular apoptosis, wherein said cellular apoptosis is the result of exposure to viral infection and cigarette smoke, said method comprising administering a therapeutically effective amount of an inhibitor to said mammal having cellular apoptosis, wherein said inhibitor prevents said cellular apoptosis, and further wherein said inhibitor is selected from the group consisting of an IL-18 inhibitor, an IL-18Rα inhibitor, and IFNγ inhibitor, a PKR inhibitor, and any combination thereof.

22. The method of claim 21, wherein said inhibitor comprises an inhibitor selected from the group consisting of an antibody, siRNA, a ribozyme, an antisense, an aptamer, a peptidomimetic, a small molecule, and any combination thereof.

23. The method of claim 22, wherein said antibody comprises an antibody selected from the group consisting of a polyclonal antibody, a monoclonal antibody, a humanized antibody, a synthetic antibody, a heavy chain antibody, a human antibody, and a biologically active fragment of an antibody.

24. The method of claim 21, wherein said mammal is a human.

25. A method of treating COPD in a mammal diagnosed with COPD, wherein said COPD comprises inflammation, alveolar remodeling, or cellular apoptosis, said method comprising administering a therapeutically effective amount of an inhibitor to said mammal having COPD, wherein said inhibitor prevents said inflammation, alveolar remodeling, or cellular apoptosis, and further wherein said inhibitor is selected from the group consisting of an IL-18 inhibitor, an IL-18Rα inhibitor, and IFNγ inhibitor, a PKR inhibitor, and any combination thereof.

26. The method of claim 25, wherein said inhibitor comprises an inhibitor selected from the group consisting of an antibody, siRNA, a ribozyme, an antisense, an aptamer, a peptidomimetic, a small molecule, and any combination thereof.

27. The method of claim 26, wherein said antibody comprises an antibody selected from the group consisting of a polyclonal antibody, a monoclonal antibody, a humanized antibody, a synthetic antibody, a heavy chain antibody, a human antibody, and a biologically active fragment of an antibody.

28. The method of claim 25, wherein said mammal is a human.
